(19) United States
(12) Patent Application Publication
(10) Pub. No.: US 2022/0361500 A1
(43) Pub. Date: Nov. 17, 2022

(54) STRIGOLACTONE DERIVATIVES AS PLANT GROWTH REGULATOR COMPOUNDS

(71) Applicant: SYNGENTA CROP PROTECTION AG, Basel (CH)

(72) Inventors: Mathilde Denise LACHIA, Stein (CH); Alexandre Franco Jean Camille LUMBROSO, Stein (CH); Pierre QUINODOZ, Stein (CH); Alain DE MESMAEKER, Stein (CH)

(73) Assignee: SYNGENTA CROP PROTECTION AG, Basel (CH)

(21) Appl. No.: 17/639,452

(22) PCT Filed: Aug. 25, 2020
(86) PCT No.: PCT/EP2020/073681

§ 371 Date: Mar. 1, 2022

(51) Int. Cl. A01N 43/90; C07D 493/04; C07D 495/04; C07D 491/04

(52) U.S. Cl. CPC A01N 43/90; C07D 493/04; C07D 495/04; C07D 491/04

(57) ABSTRACT

Compounds of the formula (I) wherein the substituents are as defined in claim 1, useful as plant growth regulators and/or seed germination promoters.

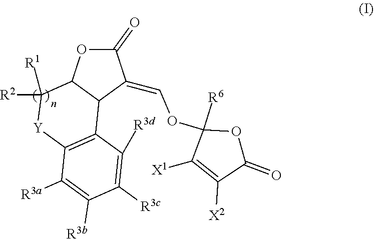

The present invention relates to relates to novel strigolactone derivatives, to processes for preparing these derivatives including intermediate compounds, to seeds comprising these derivatives, to plant growth regulator or seed germination promoting compositions comprising these derivatives and to methods of using these derivatives in controlling the growth of plants and/or promoting the germination of seeds.

Strigolactone derivatives are phytohormones which may have plant growth regulation and seed germination properties. They have previously been described in the literature. Certain known strigolactone derivatives (e.g. see WO2012/080115 and WO2016/193290) may have properties analogous to natural strigolactones, e.g. plant growth regulation and/or seed germination promotion. For such compounds to be used, in particular, for foliar applications or in seed treatment (e.g. as seed coating components), their binding affinities with the strigolactone receptor D14 are important.

The present invention relates to novel strigolactone derivatives that have improved properties. Benefits of the compounds of the present invention include improved tolerance to abiotic stress, improved seed germination, better regulation of crop growth, improved crop yield, improved nutrient use efficiency, and/or improved physical properties such as chemical, hydrolytic, physical and/or soil stability.

According to the present invention, there is provided a compound of formula (I):

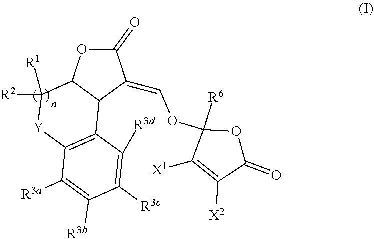

wherein

Y is O, N—R⁴, S, S(O), or S(O)₂;

n is 0 or 1;

R¹ and R² are each independently selected from hydrogen and C₁-C₄alkyl; or R¹ and R² together with the carbon atom to which they are attached form a C₃-C₆cycloalkyl group;

R^{3a}, R^{3b}, R^{3c}, R^{3d} are each independently selected from hydrogen, halogen, cyano, C₁-C₄alkyl, C₁-C₄haloalkyl, C₁-C₄alkoxy, C₁-C₄alkoxycarbonyl, C₁-C₄alkylcarbonyl, and C₃-C₆cycloalkyl, wherein each cycloalkyl moiety is optionally substituted with 1 to 3 groups represented by R⁵;

R⁴ is hydrogen, C₁-C₄alkyl, formyl, C₁-C₄alkylcarbonyl, C₁-C₄alkoxycarbonyl, C₁-C₄haloalkylcarbonyl, C₃-C₈cycloalkylcarbonyl, phenyl, —S(O)₂-C₁-C₄alkyl, or —S(O)₂-phenyl;

R⁵ is halogen, cyano, C₁-C₄alkyl, C₁-C₄haloalkyl, or C₁-C₄alkoxy;

R⁶ is hydrogen or C₁-C₄alkyl; and

X¹ and X² are each independently selected from hydrogen, halogen, cyano, C₁-C₄alkyl, and C₁-C₄alkoxy;

or a salt or an N-oxide thereof.

In a second aspect of the invention, there is provided a plant growth regulating or seed germination promoting composition, comprising the compound according to the present invention, and optionally, an agriculturally acceptable formulation adjuvant.

In a third aspect of the invention, there is provided a method for regulating the growth of plants at a locus, wherein the method comprises applying to the locus a plant growth regulating amount of the composition according to the second aspect of the invention.

In a fourth aspect of the invention, there is provided a method for promoting the germination of seeds comprising applying to the seeds, or a locus containing seeds, a seed germination promoting amount of a composition according to the second aspect of the invention.

In a fifth aspect of the invention, there is provided a method for controlling weeds, comprising applying to a locus containing weed seeds, a seed germination promoting amount of a composition according to the second aspect of the invention, allowing the seeds to germinate, and then applying to the locus a post-emergence herbicide.

In a sixth aspect of the invention, there is provided the use of a compound of Formula (I) according to the invention as a plant growth regulator or a seed germination promoter.

In a seventh aspect of the invention, there is provided a method of treating a plant propagation material comprising applying to the plant propagation material a composition according to the invention in an amount effective to promote germination and/or regulate plant growth.

In an eighth aspect of the invention, there is provided a plant propagation material treated with a compound of Formula (I) according to the invention, or a composition according to the invention.

In a ninth aspect of the invention, there is provided a seed comprising a compound of Formula (I) according to the invention.

In a tenth aspect of the invention, there is provided a method for improving the nutrient uptake of a crop, comprising applying to the plant or locus thereof, a compound of Formula (I) according to the invention, or a composition according to the invention.

Where substituents are indicated as being “optionally substituted”, this means that they may or may not carry one or more identical or different substituents, e.g., one, two or three R⁵ substituents. For example, C₁-C₄alkyl substituted by 1, 2 or 3 halogens, may include, but not be limited to, —CH₂Cl, —CHCl₂, —CCl₃, —CH₂F, —CHF₂, —CF₃, —CH₂CF₃ or —CF₂CH₃ groups. As another example, C₁-C₄alkoxy substituted by 1, 2 or 3 halogens, may include, but not limited to, CH₂ClO—, CHCl₂O—, CCl₃O—, CH₂FO—, CHF₂O—, CF₃O—, CF₃CH₂O— or CH₃CF₂O— groups.

As used herein, the term “cyano” means a —CN group.

As used herein, the term “halogen” refers to fluorine (fluoro), chlorine (chloro), bromine (bromo) or iodine (iodo).

As used herein, the term “formyl” means a —C(O)H group.

As used herein, the term “acetyl” means a —C(O)CH₃ group.

As used herein, the term “C₁-C₄alkyl” refers to a straight or branched hydrocarbon chain radical consisting solely of carbon and hydrogen atoms, containing no unsaturation, having from one to four carbon atoms, and which is attached to the rest of the molecule by a single bond. “C₁-C₃alkyl” and “C₁-C₂alkyl” are to be construed accordingly. Examples of C₁-C₄alkyl include, but are not limited to, methyl, ethyl, n-propyl, and the isomers thereof, for example, iso-propyl.

As used herein, the term “C₁-C₄haloalkyl” refers a C₁-C₄alkyl radical as generally defined above substituted by one or more of the same or different halogen atoms. The terms “C₁-C₃haloalkyl” and “C₁-C₂haloalkyl”, are to be construed accordingly. Examples of C₁-C₄haloalkyl include, but are not limited to trifluoromethyl and 2,2,2-trifluoroethyl.

As used herein, the term “C₁-C₄alkoxy” refers to a radical of the formula —ORₐ where Rₐ is a C₁-C₄alkyl radical as generally defined above. The terms “C₁-C₃alkoxy” and “C₁-C₂alkoxy” are to be construed accordingly. Examples of C₁-C₄alkoxy include, but are not limited to, methoxy, ethoxy, 1-methylethoxy (iso-propoxy), and propoxy.

As used herein, the term “C₃-C₈cycloalkyl” refers to a radical which is a monocyclic saturated ring system and which contains 3 to 8 carbon atoms. The terms “C₃-C₆cycloalkyl”, “C₅-C₆cycloalkyl” and “C₃-C₄cycloalkyl” are to be construed accordingly. Examples of C₃-C₆cycloalkyl include, but are not limited to, cyclopropyl, 1-methylcyclopropyl, 2-methylcyclopropyl, cyclobutyl, 1-methylcyclobutyl, 1,1-dimethylcyclobutyl, 2-methylcyclobutyl, 2,2-dimethylcyclobutyl, cyclopentyl and cyclohexyl.

As used herein, the term “C₁-C₄alkylcarbonyl” refers to a radical of the formula —C(O)Rₐ, where Rₐ is a C₁-C₄alkyl radical as generally defined above.

As used herein, the term “C₁-C₄haloalkylcarbonyl” refers to a radical of the formula —C(O)Rₐ, where Rₐ is a C₁-C₄haloalkyl radical as generally defined above.

As used herein, the term “C₃-C₈cycloalkylcarbonyl” refers to a radical of the formula —C(O)Rₐ, where Rₐ is a C₃-C₈cycloalkyl radical as generally defined above.

As used herein, the term “C₁-C₄alkoxycarbonyl” refers to a radical of the formula —C(O)ORₐ, where Rₐ is a C₁-C₄alkyl radical as generally defined above.

The presence of one or more possible stereogenic elements in a compound of formula (I) means that the compounds may occur in optically isomeric forms, i.e., enantiomeric or diastereomeric forms. Also, atropisomers may occur as a result of restricted rotation about a single bond. Formula (I) is intended to include all those possible isomeric forms and mixtures thereof. The present invention includes all those possible isomeric forms and mixtures thereof for a compound of formula (I). Likewise, formula (I) is intended to include all possible tautomers. The present invention includes all possible tautomeric forms for a compound of formula (I). In each case, the compounds of formula (I) according to the invention are in free form, in oxidized form as an N-oxide, or in salt form, e.g., an agronomically usable salt form. N-oxides are oxidized forms of tertiary amines or oxidized forms of nitrogen-containing heteroaromatic compounds. They are described for instance in the book “Heterocyclic N-oxides” by A. Albini and S. Pietra, CRC Press, Boca Raton (1991).

The following list provides definitions, including preferred definitions, for substituents Y, R¹, R², R^{3a}, R^{3b}, R^{3c}, R^{3d}, R⁴, R⁵, R⁶, X¹ and X², with reference to compounds of formula (I). For any one of these substituents, any of the definitions given below may be combined with any definition of any other substituent given below or elsewhere in this document.

Y is O, N—R⁴, S, S(O), or S(O)₂. Preferably, Y is O, S, or S(O). In one set of embodiments, Y is O. In another set of embodiments, Y is S. In a further set of embodiments, Y is S(O).

n is 0 or 1. In one set of embodiments, n is 0, in another set of embodiments, n is 1.

In one set of embodiments, when n is 0, Y is O, N—R⁴, S, S(O), or S(O)₂. In another set of embodiments, when n is 1, Y is O.

R¹ and R² are each independently selected from hydrogen and C₁-C₄alkyl; or R¹ and R² together with the carbon atom to which they are attached form a C₃-C₆cycloalkyl group. Preferably, R¹ and R² are each independently selected from hydrogen and C₁-C₃alkyl; or R¹ and R² together with the carbon atom to which they are attached form a C₃-C₆cycloalkyl group. More preferably, R¹ and R² are each independently selected from hydrogen, methyl and ethyl; or R¹ and R² together with the carbon atom to which they are attached form a C₅-C₆cycloalkyl group. Even more preferably, R¹ and R² are each independently selected from hydrogen, methyl and ethyl, and most preferably, hydrogen and methyl.

In one set of embodiments, R¹ and R² are both hydrogen. In another set of embodiments, R¹ and R² are both methyl.

R^{3a}, R^{3b}, R^{3c}, R^{3d} are each independently selected from hydrogen, halogen, cyano, C₁-C₄alkyl, C₁-C₄haloalkyl, C₁-C₄alkoxy, C₁-C₄alkoxycarbonyl, C₁-C₄alkylcarbonyl, and C₃-C₆cycloalkyl, wherein each cycloalkyl moiety is optionally substituted with 1 to 3 groups represented by R⁵. Preferably, R^{3a}, R^{3b}, R^{3c}, R^{3d} are each independently selected from hydrogen, halogen, cyano, C₁-C₄alkyl, C₁-C₄haloalkyl, C₁-C₄alkoxy, C₁-C₄alkoxycarbonyl, and C₁-C₄alkylcarbonyl. More preferably, R^{3a}, R^{3b}, R^{3c}, R^{3d} are each independently selected from hydrogen, halogen, cyano, C₁-C₃alkyl, C₁-C₃haloalkyl, C₁-C₃alkoxy, and C₁-C₃alkylcarbonyl. More preferably still, R^{3a}, R^{3b}, R^{3c}, R^{3d} are each independently selected from hydrogen, chloro, fluoro, methyl, ethyl, isopropyl, trifluoromethyl, 2,2,2-trifluoroethyl, methoxy, isopropoxy and acetyl. Even more preferably, R^{3a}, R^{3b}, R^{3c}, R^{3d} are each independently selected from hydrogen, chloro, fluoro, methyl, trifluoromethyl or methoxy. Most preferably, R^{3a}, R^{3b}, R^{3c}, R^{3d} are all hydrogen.

R⁴ is hydrogen, C₁-C₄alkyl, formyl, C₁-C₄alkylcarbonyl, C₁-C₄alkoxycarbonyl, C₁-C₄haloalkylcarbonyl, C₃-C₈cycloalkylcarbonyl, phenyl, —S(O)₂-C₁-C₄alkyl, or —S(O)₂-phenyl. Preferably, R⁴ is hydrogen, C₁-C₄alkyl, formyl, C₁-C₄alkylcarbonyl, C₁-C₄alkoxycarbonyl, C₁-C₄haloalkylcarbonyl, C₃-C₆cycloalkylcarbonyl, phenyl, —S(O)₂-methyl, or —S(O)₂-phenyl. More preferably, R⁴ is hydrogen, C₁-C₃alkyl, formyl, C₁-C₃alkylcarbonyl, C₁-C₃alkoxycarbonyl, C₁-C₃haloalkylcarbonyl, C₃-C₆cycloalkylcarbonyl, phenyl, —S(O)₂-methyl, or —S(O)₂-phenyl. Even more preferably, R⁴ is hydrogen, C₁-C₃alkyl, formyl, C₁-C₃alkylcarbonyl, C₁-C₃alkoxycarbonyl, C₁-C₃haloalkylcarbonyl, phenyl, or —S(O)₂-methyl. More preferably still, R⁴ is C₁-C₃alkyl, formyl, C₁-C₃alkylcarbonyl, C₁-C₃haloalkylcarbonyl, phenyl, or —S(O)₂-methyl. Even more preferably still, R⁴ is methyl, ethyl, formyl, acetyl, phenyl or —S(O)₂-methyl.

In one set of embodiments, R⁴ is —S(O)₂-C₁-C₄alkyl, preferably R⁴ is —S(O)₂C₁-C₃alkyl, and more preferably, R⁴ is —S(O)₂methyl.

R⁵ is halogen, cyano, C₁-C₄alkyl, C₁-C₄haloalkyl, or C₁-C₄alkoxy. Preferably, R⁵ is halogen, cyano, C₁-C₃alkyl, C₁-C₃haloalkyl, or C₁-C₃alkoxy. More preferably, R⁵ is chloro, fluoro, methyl, ethyl, n-propyl, isopropyl, trifluoromethyl, 2,2,2-trifluoroethyl, methoxy, ethoxy or isopropoxy. Even more preferably, R⁵ is chloro, fluoro, methyl, ethyl, isopropyl, trifluoromethyl or methoxy. More preferably still, R⁵ is chloro, fluoro, or methyl.

R⁶ is hydrogen or C₁-C₄alkyl. Preferably, R⁶ is hydrogen or C₁-C₃alkyl. More preferably, R⁶ is hydrogen, methyl or ethyl, even more preferably, hydrogen or methyl. Most preferably, R⁶ is hydrogen.

X¹ and X² are each independently selected from hydrogen, halogen, cyano, C₁-C₄alkyl, and C₁-C₄alkoxy. Preferably, X¹ and X² are each independently selected from hydrogen, halogen, cyano, C₁-C₃alkyl, and C₁-C₃alkoxy. More preferably, X¹ and X² are each independently selected from hydrogen, halogen, C₁-C₃alkyl, and C₁-C₃alkoxy. Even more preferably, X¹ and X² are each independently selected from hydrogen, halogen, methyl, ethyl, methoxy, ethoxy and isopropoxy. More preferably still, X¹ and X² are each independently selected from hydrogen, chloro, fluoro, methyl, ethyl, methoxy and ethoxy.

In one set of embodiments, X¹ is selected from hydrogen, chloro, methyl and methoxy, preferably hydrogen and methyl.

In one set of embodiments, X² is selected from methyl and ethyl, preferably methyl.

In a further set of embodiments, when X² is methyl, X¹ is hydrogen. In another set of embodiments, both X¹ and X² are methyl.

In a compound of formula (I) according to the present invention, preferably:
Y is O, N—R⁴, S, S(O), or S(O)₂;
n is 0 or 1;
R¹ and R² are each independently selected from hydrogen and C₁-C₄alkyl; or R¹ and R² together with the carbon atom to which they are attached form a C₅-C₆cycloalkyl group;
R^{3a}, R^{3b}, R^{3c}, R^{3d} are each independently selected from hydrogen, halogen, cyano, methyl, trifluoromethyl, methoxy, methoxycarbonyl, and acetyl;
R⁴ is —S(O)₂C₁-C₃alkyl;
R⁶ is hydrogen; and
X¹ and X² are each independently selected from hydrogen and methyl.

In one set of embodiments, Y is O, N—R⁴, S, S(O), or S(O)₂;

n is 0 or 1;

R¹ and R² are each independently selected from hydrogen and C₁-C₄alkyl;

R^{3a}, R^{3b}, R^{3c}, R^{3d} are all hydrogen;

R⁴ is —S(O)₂-methyl;

R⁶ is hydrogen or C₁-C₄ alkyl; and

X¹ and X² are each independently selected from hydrogen, chloro, methyl and methoxy.

In one set of embodiments, Y is O, N—R⁴, S, S(O), or S(O)₂;

n is 0 or 1;

R¹ and R² are each independently selected from hydrogen and methyl;

R^{3a}, R^{3b}, R^{3c}, R^{3d} are all hydrogen;

R⁴ is —S(O)₂-methyl;

R⁶ is hydrogen; and

X¹ and X² are independently selected from hydrogen and methyl.

In one set of embodiments, Y is O, N—R⁴, S, S(O), or S(O)₂;

n is 0;

R¹ and R² are each independently selected from hydrogen and methyl;

R^{3a}, R^{3b}, R^{3c}, R^{3d} are all hydrogen;

R⁴ is —S(O)₂-methyl;

R⁶ is hydrogen;

X¹ is hydrogen or methyl; and

X² is methyl.

In one set of embodiments, Y is O;

n is 1;

R¹ and R² are each independently selected from hydrogen and methyl;

R^{3a}, R^{3b}, R^{3c}, R^{3d} are all hydrogen;

R⁶ is hydrogen;

X¹ is hydrogen; and

X² is methyl.

Preferably, the compound according to formula (I) is selected from:
(1E)-1-[(4-methyl-5-oxo-2H-furan-2-yl)oxymethylene]-3a,8b-dihydrofuro[2,3-b]benzofuran-2-one (I-1);
(1E)-1-[(3,4-dimethyl-5-oxo-2H-furan-2-yl)oxymethylene]-3a,8b-dihydrofuro[2,3-b]benzofuran-2-one (I-2);
(1E)-1-[(4-methyl-5-oxo-2H-furan-2-yl)oxymethylene]-3a,8b-dihydrobenzothiopheno[2,3-b]furan-2-one (I-7);
(1E)-1-[(3,4-dimethyl-5-oxo-2H-furan-2-yl)oxymethylene]-3a,8b-dihydrobenzothiopheno[2,3-b]furan-2-one (I-8);
(1E)-1-[(4-methyl-5-oxo-2H-furan-2-yl)oxymethylene]-4-oxo-3a,8b-dihydrobenzothiopheno[2,3-b]furan-2-one (I-13);
(1E)-1-[(3,4-dimethyl-5-oxo-2H-furan-2-yl)oxymethylene]-3a,8b-dihydrofuro[2,3-b]benzofuran-2-one (I-14);
(1E)-1-[(4-methyl-5-oxo-2H-furan-2-yl)oxymethylene]-4,4-dioxo-3a,8b-dihydrobenzothiopheno[2,3-b]furan-2-one (I-19);
(1E)-1-[(3,4-dimethyl-5-oxo-2H-furan-2-yl)oxymethylene]-4,4-dioxo-3a,8b-dihydrobenzothiopheno[2,3-b]furan-2-one (I-20);
(1E)-1-[(4-methyl-5-oxo-2H-furan-2-yl)oxymethylene]-4-methylsulfonyl-3a,8b-dihydrofuro[2,3-b]indol-2-one (I-25);
(1E)-1-[(3,4-dimethyl-5-oxo-2H-furan-2-yl)oxymethylene]-4-methylsulfonyl-3a,8b-dihydrofuro[2,3-b]indol-2-one (I-26);
(1E)-1-[(4-methyl-5-oxo-2H-furan-2-yl)oxymethylene]-4,9b-dihydro-3aH-furo[2,3-c]chromen-2-one (I-31); and
(1E)-4,4-dimethyl-1-[(4-methyl-5-oxo-2H-furan-2-yl)oxymethylene]-3a,9b-dihydrofuro[2,3-c]chromen-2-one (I-37).

More preferably, the compound according to formula (I) is selected from:
(1E)-1-[(4-methyl-5-oxo-2H-furan-2-yl)oxymethylene]-3a,8b-dihydrofuro[2,3-b]benzofuran-2-one (I-1);
(1E)-1-[(3,4-dimethyl-5-oxo-2H-furan-2-yl)oxymethylene]-3a,8b-dihydrofuro[2,3-b]benzofuran-2-one (I-2);
(1E)-1-[(3,4-dimethyl-5-oxo-2H-furan-2-yl)oxymethylene]-3a,8b-dihydrobenzothiopheno[2,3-b]furan-2-one (I-8);
(1E)-1-[(4-methyl-5-oxo-2H-furan-2-yl)oxymethylene]-4-oxo-3a,8b-dihydrobenzothiopheno[2,3-b]furan-2-one (I-13); and
(1E)-1-[(4-methyl-5-oxo-2H-furan-2-yl)oxymethylene]-4,9b-dihydro-3aH-furo[2,3-c]chromen-2-one (I-31).

Compounds of the present invention can be made as shown in the following schemes, in which, unless otherwise stated, the definition of each variable is as defined above for a compound of formula (I).

Compounds of formula (I) may be prepared from compounds of formula (II) by reaction with a compound of formula (III) and compound (A or B) in the presence of a base such potassium tert-butylate or sodium tert-butylate, optionally in the presence of a crown ether to activate the base. The reaction can also be carried out in the presence of a catalytic or stoichiometric amount of iodine salt, such as potassium iodide or tetrabutyl ammonium iodide.

Alternatively, compound (Ia), wherein Y is S(O) or S(O)₂, can be prepared from a compound of formula (I) wherein Y is S, by oxidation with a reagent such as mCPBA or oxone. This is shown in Scheme 1 below.

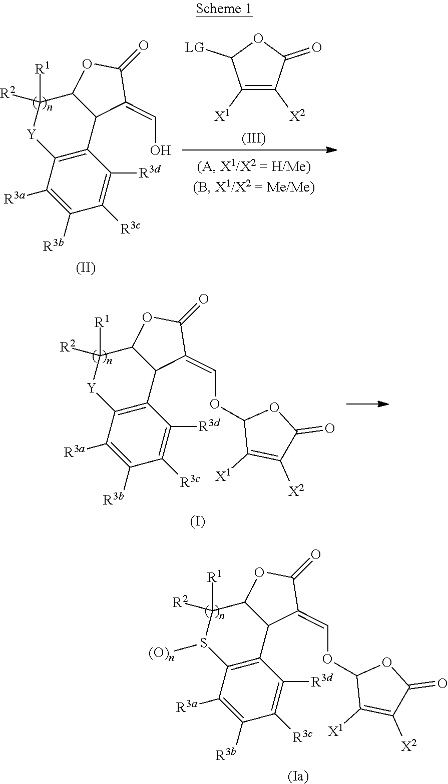

Compounds of formula (II) may be prepared from a compound of formula (IV) via reaction with a formic ester derivative such as the methyl formate in the presence of a base such as lithium diisopropylamide, potassium tert-butylate or sodium tert-butylate.

Alternatively, compounds of formula (II) may be prepared from a compound of formula (V) wherein R is methyl, via hydrolysis with an acid such as aqueous hydrogen chloride. Compounds of formula (V) wherein R is methyl, may be prepared from a compound of formula (IV) via reaction with Bredereck's reagent (tert-butoxybis(dimethylamino)methane). This is shown in Scheme 2 below.

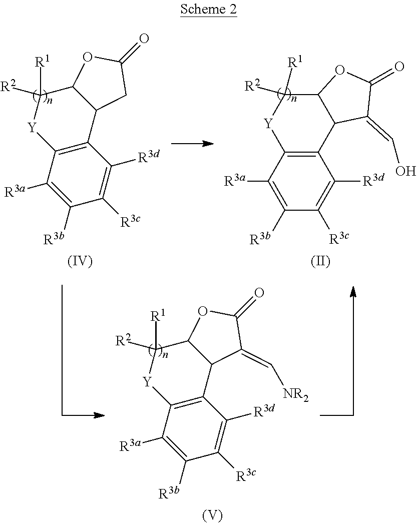

Compounds of formula (IV) may be prepared from a compound of formula (VII) via a reduction reaction using an organic or inorganic acid such as ammonium chloride and a metal source such as zinc. Compounds of formula (VII) may be prepared from a compound of formula (VI) via a Baeyer-Villiger reaction using a peroxide such as magnesium monoperoxyphthalate (MMPP), hydrogen peroxide or mCPBA optionally in the presence of an acid such as acetic acid.

Alternatively, compound of formula (IV) may be prepared form compound of formula (VIII) via a Bayer-Villiger reaction using a peroxide such as magnesium monoperoxyphthalate (MMPP), hydrogen peroxide or mCPBA, optionally in the presence of an acid such as acetic acid. Compound of formula (VIII) may be prepared from compound of formula (VI) via a reduction reaction using an acid such as ammonium chloride and a metal such as zinc. This is shown in Scheme 3 below.

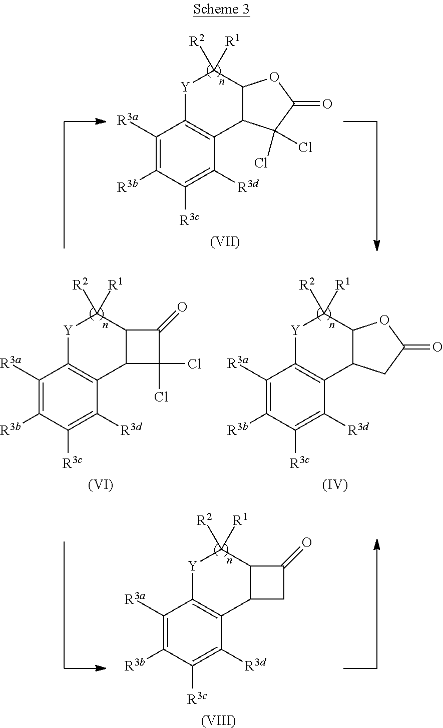

Compounds of formula (VI) may be prepared form commercially available compounds of formula (IX) via a [2+2] cycloaddition reaction with a ketene such as dichloroketene. This is shown in Scheme 4 below.

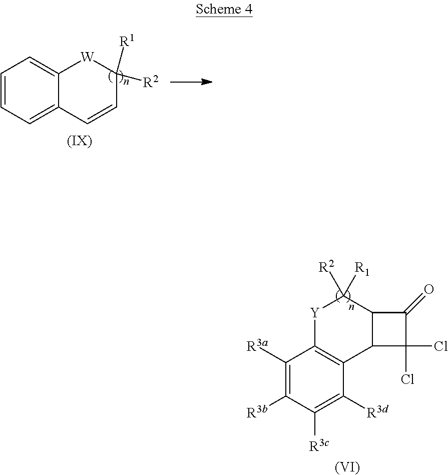

Alternatively, compounds of formula (VIII) can be prepared from a compound of formula (X) via a [2+2] cycloaddition reaction with a keteniminium salt using a base such as sym-collidine or 2-halogeno pyridine (e.g. 2-fluoropyridine) and triflic anhydride. Compounds of formula (X) can be prepared from a compound of formula (XI), wherein R is C₁-C₄alkyl, C₃-C₆alkenyl, or the two R groups along with the nitrogen atom to which they are attached, are joined to form a 5- to 7-membered cycloalkyl ring; X is Br, Cl or I, and a vinyl metal derivative, wherein [M] can be a boron or a tin derivative, in the presence of a suitable catalyst/ligand system, often a palladium (0) complex. This is shown in Scheme 5 below.

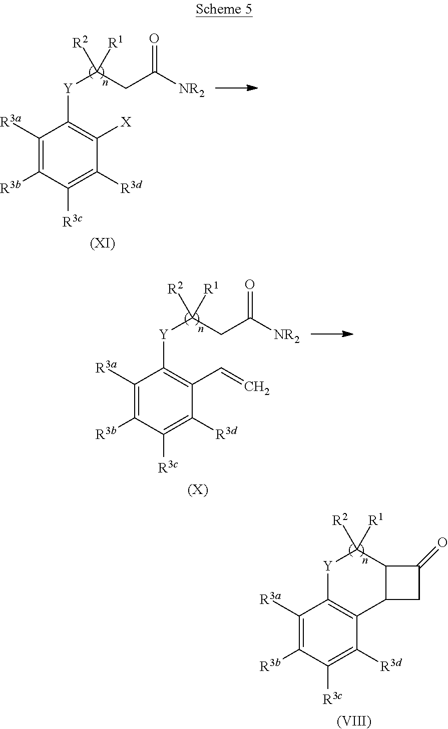

Compounds of formula (XI) can be prepared from known compounds of formula (XII) and (XIII) wherein LG is a leaving group such as Br or I, using a base such as triethylamine amine or sodium hydride. This is shown in Scheme 6 below.

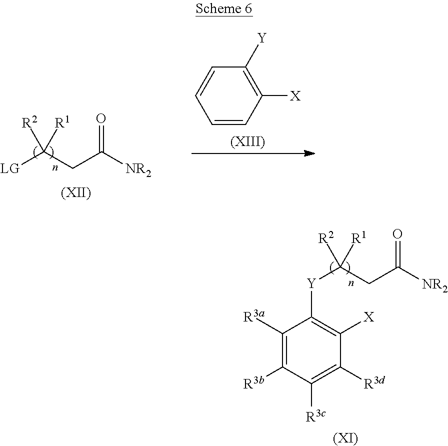

Table 1 below illustrates examples of individual compounds of formula (I) according to the invention.

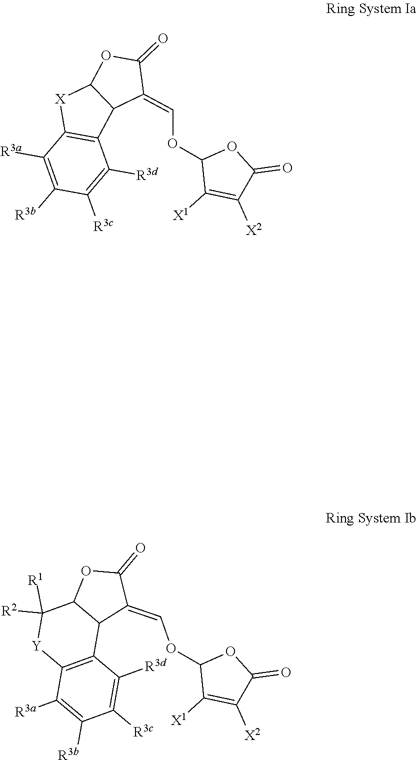

TABLE 1
Individual compounds of formula (I) according
to the invention wherein R^{3a}, R^{3b}, R^{3c} and
R^{3d} are each independently hydrogen, and
Y, R¹, R², X¹, and X² are as described below:

| Cpd | Ring | | | | | |
|---|---|---|---|---|---|---|
| No. | system | Y | R¹ | R² | X¹ | X² |
| I-1 | Ia | O | — | — | H | Me |
| I-2 | Ia | O | — | — | Me | Me |
| I-3 | Ia | O | — | — | OMe | Me |
| I-4 | Ia | O | — | — | Cl | Me |
| I-5 | Ia | O | — | — | H | Et |
| I-6 | Ia | O | — | — | Me | Et |
| I-7 | Ia | S | — | — | H | Me |
| I-8 | Ia | S | — | — | Me | Me |
| I-9 | Ia | S | — | — | OMe | Me |
| I-10 | Ia | S | — | — | Cl | Me |
| I-11 | Ia | S | — | — | H | Et |
| I-12 | Ia | S | — | — | Me | Et |
| I-13 | Ia | S(O) | — | — | H | Me |
| I-14 | Ia | S(O) | — | — | Me | Me |
| I-15 | Ia | S(O) | — | — | OMe | Me |
| I-16 | Ia | S(O) | — | — | Cl | Me |
| I-17 | Ia | S(O) | — | — | H | Et |
| I-18 | Ia | S(O) | — | — | Me | Et |
| I-19 | Ia | S(O)₂ | — | — | H | Me |
| I-20 | Ia | S(O)₂ | — | — | Me | Me |
| I-21 | Ia | S(O)₂ | — | — | OMe | Me |
| I-22 | Ia | S(O)₂ | — | — | Cl | Me |
| I-23 | Ia | S(O)₂ | — | — | H | Et |
| I-24 | Ia | S(O)₂ | — | — | Me | Et |
| I-25 | Ia | N—SO₂Me | — | — | H | Me |
| I-26 | Ia | N—SO₂Me | — | — | Me | Me |
| I-27 | Ia | N—SO₂Me | — | — | OMe | Me |
| I-28 | Ia | N—SO₂Me | — | — | Cl | Me |
| I-29 | Ia | N—SO₂Me | — | — | H | Et |
| I-30 | Ia | N—SO₂Me | — | — | Me | Et |
| I-31 | Ib | O | H | H | H | Me |
| I-32 | Ib | O | H | H | Me | Me |
| I-33 | Ib | O | H | H | OMe | Me |
| I-34 | Ib | O | H | H | Cl | Me |
| I-35 | Ib | O | H | H | H | Et |
| I-36 | Ib | O | H | H | Me | Et |
| I-37 | Ib | O | Me | Me | H | Me |
| I-38 | Ib | O | Me | Me | Me | Me |
| I-39 | Ib | O | Me | Me | OMe | Me |
| I-40 | Ib | O | Me | Me | Cl | Me |
| I-41 | Ib | O | Me | Me | H | Et |
| I-42 | Ib | O | Me | Me | Me | Et |

The present invention provides a method of improving the tolerance of a plant to abiotic stress, wherein the method comprises applying to the plant, plant part, plant propagation material, or plant growing locus a compound, composition or mixture according to the present invention.

The present invention provides a method for regulating or improving the growth of a plant, wherein the method comprises applying to the plant, plant part, plant propagation material, or plant growing locus a compound, composition or mixture according to the present invention. In one embodiment, plant growth is regulated or improved when the plant is subject to abiotic stress conditions.

The present invention also provides a method for improving seed germination of a plant, and especially the present invention provides a method for improving seed germination of a plant under cold stress conditions, comprising applying to the seed, or a locus containing seeds, a compound, a composition or mixture according to the present invention.

The present invention also provides a method for safening a plant against phytotoxic effects of chemicals, comprising applying to the plant, plant part, plant propagation material, or plant growing locus a compound, a composition or mixture according to the present invention.

The present invention also provides a method for inducing/increasing leaf senescence in crops of useful plants, said method comprising applying to the plant, plant part, plant propagation material, or plant growing locus a compound, a composition or mixture according to the present invention. In particular, there is provided a method for inducing/increasing leaf senescence in corn, said method comprising applying to the corn plant, plant part, plant propagation material, or plant growing locus a compound, a composition or mixture according to the present invention.

According to the present invention, “regulating or improving the growth of a crop” means an improvement in plant vigour, an improvement in plant quality, improved tolerance to stress factors, and/or improved input use efficiency.

An ‘improvement in plant vigour’ means that certain traits are improved qualitatively or quantitatively when compared with the same trait in a control plant which has been grown under the same conditions in the absence of the method of the invention. Such traits include, but are not limited to, early and/or improved germination, improved emergence, the ability to use fewer seeds, increased root growth, a more developed root system, increased root nodulation, increased shoot growth, increased tillering, stronger tillers, more productive tillers, increased or improved plant stand, less plant verse (lodging), an increase and/or improvement in plant height, an increase in plant weight (fresh or dry), bigger leaf blades, greener leaf colour, increased pigment content, increased photosynthetic activity, earlier flowering, longer panicles, early grain maturity, increased seed, fruit or pod size, increased pod or ear number, increased seed number per pod or ear, increased seed mass, enhanced seed filling, fewer dead basal leaves, delay of senescence, improved vitality of the plant, increased levels of amino acids in storage tissues and/or fewer inputs needed (e.g. less fertiliser, water and/or labour needed). A plant with improved vigour may have an increase in any of the aforementioned traits or any combination or two or more of the aforementioned traits.

An ‘improvement in plant quality’ means that certain traits are improved qualitatively or quantitatively when compared with the same trait in a control plant which has been grown under the same conditions in the absence of the method of the invention. Such traits include, but are not limited to, improved visual appearance of the plant, reduced ethylene (reduced production and/or inhibition of reception), improved quality of harvested material, e.g. seeds, fruits, leaves, vegetables (such improved quality may manifest as improved visual appearance of the harvested material), improved carbohydrate content (e.g. increased quantities of sugar and/or starch, improved sugar acid ratio, reduction of reducing sugars, increased rate of development of sugar), improved protein content, improved oil content and composition, improved nutritional value, reduction in anti-nutritional compounds, improved organoleptic properties (e.g. improved taste) and/or improved consumer health benefits (e.g. increased levels of vitamins and anti-oxidants), improved post-harvest characteristics (e.g. enhanced shelf-life and/or storage stability, easier processability, easier extraction of compounds), more homogenous crop development (e.g. synchronised germination, flowering and/or fruiting of plants), and/or improved seed quality (e.g. for use in following seasons). A plant with improved quality may have an increase in any of the aforementioned traits or any combination or two or more of the aforementioned traits.

An ‘improved tolerance to stress factors’ means that certain traits are improved qualitatively or quantitatively when compared with the same trait in a control plant which has been grown under the same conditions in the absence of the method of the invention. Such traits include, but are not limited to, an increased tolerance and/or resistance to abiotic stress factors which cause sub-optimal growing conditions such as drought (e.g. any stress which leads to a lack of water content in plants, a lack of water uptake potential or a reduction in the water supply to plants), cold exposure, heat exposure, osmotic stress, UV stress, flooding, increased salinity (e.g. in the soil), increased mineral exposure, ozone exposure, high light exposure and/or limited availability of nutrients (e.g. nitrogen and/or phosphorus nutrients). A plant with improved tolerance to stress factors may have an increase in any of the aforementioned traits or any combination or two or more of the aforementioned traits. In the case of drought and nutrient stress, such improved tolerances may be due to, for example, more efficient uptake, use or retention of water and nutrients. In particular, the compounds or compositions of the present invention are useful to improve tolerance to drought stress.

An ‘improved input use efficiency’ means that the plants are able to grow more effectively using given levels of inputs compared to the growth of control plants which are grown under the same conditions in the absence of the method of the invention. In particular, the inputs include, but are not limited to fertiliser (such as nitrogen, phosphorous, potassium, and micronutrients), light and water. A plant with improved input use efficiency may have an improved use of any of the aforementioned inputs or any combination of two or more of the aforementioned inputs.

Other effects of regulating or improving the growth of a crop include a decrease in plant height, or reduction in tillering, which are beneficial features in crops or conditions where it is desirable to have less biomass and fewer tillers.

Any or all of the above crop enhancements may lead to an improved yield by improving e.g. plant physiology, plant growth and development and/or plant architecture. In the context of the present invention ‘yield’ includes, but is not limited to, (i) an increase in biomass production, grain yield, starch content, oil content and/or protein content, which may result from (a) an increase in the amount produced by the plant per se or (b) an improved ability to harvest plant matter, (ii) an improvement in the composition of the harvested material (e.g. improved sugar acid ratios, improved oil composition, increased nutritional value, reduction of anti-nutritional compounds, increased consumer health benefits) and/or (iii) an increased/facilitated ability to harvest the crop, improved processability of the crop and/or better storage stability/shelf life. Increased yield of an agricultural plant means that, where it is possible to take a quantitative measurement, the yield of a product of the respective plant is increased by a measurable amount over the yield of the same product of the plant produced under the same conditions, but without application of the present invention. According to the present invention, it is preferred that the yield be increased by at least 0.5%, more preferred at least 1%, even more preferred at least 2%, still more preferred at least 4%, preferably 5% or even more.

Any or all of the above crop enhancements may also lead to an improved utilisation of land, i.e. land which was previously unavailable or sub-optimal for cultivation may become available. For example, plants which show an increased ability to survive in drought conditions, may be able to be cultivated in areas of sub-optimal rainfall, e.g. perhaps on the fringe of a desert or even the desert itself.

In one aspect of the present invention, crop enhancements are made in the substantial absence of pressure from pests and/or diseases and/or abiotic stress. In a further aspect of the present invention, improvements in plant vigour, stress tolerance, quality and/or yield are made in the substantial absence of pressure from pests and/or diseases. For example, pests and/or diseases may be controlled by a pesticidal treatment that is applied prior to, or at the same time as, the method of the present invention. In a still further aspect of the present invention, improvements in plant vigour, stress tolerance, quality and/or yield are made in the absence of pest and/or disease pressure. In a further embodiment, improvements in plant vigour, quality and/or yield are made in the absence, or substantial absence, of abiotic stress.

The following mixtures of the compounds of formula (I) with active ingredients are preferred. The abbreviation “TX” means one compound selected from the group of compounds I-1 to I-42 described in Table 1, and the compounds described in Table 3 (below):

a compound selected from the group of substances consisting of petroleum oils+TX, 1,1-bis(4-chloro-phenyl)-2-ethoxyethanol+TX, 2,4-dichlorophenyl benzenesulfonate+TX, 2-fluoro-N-methyl-N-1-naphthylacetamide+TX, 4-chlorophenyl phenyl sulfone+TX, acetoprole+TX, aldoxycarb+TX, amidithion+TX, amidothioate+TX, amiton+TX, amiton hydrogen oxalate+TX, amitraz+TX, aramite+TX, arsenous oxide+TX, azobenzene+TX, azothoate+TX, benomyl+TX, benoxa-fos+TX, benzyl benzoate+TX, bixafen+TX, brofenvalerate+TX, bromo-cyclen+TX, bromophos+TX, bromopropylate+TX, buprofezin+TX, butocarboxim+TX, butoxycarboxim+TX, butylpyridaben+TX, calcium polysulfide+TX, camphechlor+TX, carbanolate+TX, carbophenothion+TX, cymiazole+TX, chino-methionat+TX, chlorbenside+TX, chlordimeform+TX, chlordimeform hydrochloride+TX, chlorfenethol+TX, chlorfenson+TX, chlorfensulfide+TX, chlorobenzilate+TX, chloromebuform+TX, chloromethiuron+TX, chloropropylate+TX, chlorthiophos+TX, cinerin 1+TX, cinerin 11+TX, cinerins+TX, closantel+TX, coumaphos+TX, crotamiton+TX, crotoxyphos+TX, cufraneb+TX, cyanthoate+TX, DCPM+TX, DDT+TX, demephion+TX, demephion-O+TX, demephion-S+TX, demeton-methyl+TX, demeton-O+TX, demeton-O-methyl+TX, demeton-S+TX, demeton-S-methyl+TX, demeton-S-methylsulfon+TX, dichlofluanid+TX, dichlorvos+TX, dicliphos+TX, dienochlor+TX, dimefox+TX, dinex+TX, dinex-diclexine+TX, dinocap-4+TX, dinocap-6+TX, dinocton+TX, dino-penton+TX, dinosulfon+TX, dinoterbon+TX, dioxathion+TX, diphenyl sulfone+TX, disulfiram+TX, DNOC+TX, dofenapyn+TX, doramectin+TX, endothion+TX, eprinomectin+TX, ethoate-methyl+TX, etrimfos+TX, fenazaflor+TX, fenbutatin oxide+TX, fenothiocarb+TX, fenpyrad+TX, fen-pyroximate+TX, fenpyrazamine+TX, fenson+TX, fentrifanil+TX, flubenzimine+TX, flucycloxuron+TX, fluenetil+TX, fluorbenside+TX, FMC 1137+TX, formetanate+TX, formetanate hydrochloride+TX, formparanate+TX, gamma-HCH+TX, glyodin+TX, halfenprox+TX, hexadecyl cyclopropanecarboxylate+TX, isocarbophos+TX, jasmolin I+TX, jasmolin II+TX, jodfenphos+TX, lindane+TX, malonoben+TX, mecarbam+TX, mephosfolan+TX, mesulfen+TX, methacrifos+TX, methyl bromide+TX, metolcarb+TX, mexacarbate+TX, milbemycin oxime+TX, mipafox+TX, monocrotophos+TX, morphothion+TX, moxidectin+TX, naled+TX, 4-chloro-2-(2-chloro-2-methyl-propyl)-5-[(6-iodo-3-pyridyl)methoxy]pyridazin-3-one+TX, nifluridide+TX, nikkomycins+TX, nitrilacarb+TX, nitrilacarb 1:1 zinc chloride complex+TX, omethoate+TX, oxydeprofos+TX, oxydisulfoton+TX, pp′-DDT+TX, parathion+TX, permethrin+TX, phenkapton+TX, phosalone+TX, phosfolan+TX, phosphamidon+TX, polychloroterpenes+TX, polynactins+TX, proclonol+TX, promacyl+TX, propoxur+TX, prothidathion+TX, prothoate+TX, pyrethrin I+TX, pyrethrin II+TX, pyrethrins+TX, pyridaphenthion+TX, pyrimitate+TX, quinalphos+TX, quintiofos+TX, R-1492+TX, phosglycin+TX, rotenone+TX, schradan+TX, sebufos+TX, selamectin+TX, sophamide+TX, SSI-121+TX, sulfiram+TX, sulfluramid+TX, sulfotep+TX, sulfur+TX, diflovidazin+TX, tau-fluvalinate+TX, TEPP+TX, terbam+TX, tetradifon+TX, tetrasul+TX, thiafenox+TX, thiocarboxime+TX, thiofanox+TX, thiometon+TX, thioquinox+TX, thuringiensin+TX, triamiphos+TX, triarathene+TX, triazophos+TX, triazuron+TX, trifenofos+TX, trinactin+TX, vamidothion+TX, vaniliprole+TX, bethoxazin+TX, copper dioctanoate+TX, copper sulfate+TX, cybutryne+TX, dichlone+TX, dichlorophen+TX, endothal+TX, fentin+TX, hydrated lime+TX, nabam+TX, quinoclamine+TX, quinonamid+TX, simazine+TX, triphenyltin acetate+TX, triphenyltin hydroxide+TX, crufomate+TX, piperazine+TX, thiophanate+TX, chloralose+TX, fenthion+TX, pyridin-4-amine+TX, strychnine+TX, 1-hydroxy-1H-pyridine-2-thione+TX, 4-(quinoxalin-2-ylamino)benzenesulfonamide+TX, 8-hydroxyquinoline sulfate+TX, bronopol+TX, copper hydroxide+TX, cresol+TX, dipyrithione+TX, dodicin+TX, fenaminosulf+TX, formaldehyde+TX, hydrargaphen+TX, kasugamycin+TX, kasugamycin hydrochloride hydrate+TX, nickel bis(dimethyldithiocarbamate)+TX, nitrapyrin+TX, octhilinone+TX, oxolinic acid+TX, oxytetracycline+TX, potassium hydroxyquinoline sulfate+TX, probenazole+TX, streptomycin+TX, streptomycin sesquisulfate+TX, tecloftalam+TX, thiomersal+TX, Adoxophyes orana GV+TX, Agrobacterium radiobacter+TX, Amblyseius spp.+TX, Anagrapha falcifera NPV+TX, Anagrus atomus+TX, Aphelinus abdominalis+TX, Aphidius colemani+TX, Aphidoletes aphidimyza+TX, Autographa californica NPV+TX, Bacillus sphaericus Neide+TX, Beauveria brongniartii+TX, Chrysoperla carnea+TX, Cryptolaemus montrouzieri+TX, Cydia pomonella GV+TX, Dacnusa sibirica+TX, Diglyphus isaea+TX, Encarsia formosa+TX, Eretmocerus eremicus+TX, Heterorhabditis bacteriophora and H. megidis+TX, Hippodamia convergens+TX, Leptomastix dactylopii+TX, Macrolophus caliginosus+TX, Mamestra brassicae NPV+TX, Metaphycus helvolus+TX, Metarhizium anisopliae var. acridum+TX, Metarhizium anisopliae var. anisopliae+TX, Neodiprion sertifer NPV and N. lecontei NPV+TX, Orius spp.+TX, Paecilomyces fumosoroseus+TX, Phytoseiulus persimilis+TX, Steinernema bibionis+TX, Steinernema carpocapsae+TX, Steinernema feltiae+TX, Steinernema glaseri+TX, Steinernema riobrave+TX, Steinernema riobravis+TX, Steinernema scapterisci+TX, Steinernema spp.+TX, Trichogramma spp.+TX, Typhlodromus occidentalis+TX, Verticillium lecanii+TX, apholate+TX, bisazir+TX, busulfan+TX, dimatif+TX, hemel+TX, hempa+TX, metepa+TX, methiotepa+TX, methyl apholate+TX, morzid+TX, penfluron+TX, tepa+TX, thiohempa+TX, thiotepa+TX, tretamine+TX, uredepa+TX, (E)-dec-5-en-1-yl acetate with (E)-dec-5-en-1-ol+TX, (E)-tridec-4-en-1-yl acetate+TX, (E)-6-methylhept-2-en-4-ol+TX, (E,Z)-tetradeca-4,10-dien-1-yl acetate+TX, (Z)-dodec-7-en-1-yl acetate+TX, (Z)-hexadec-11-enal+TX, (Z)-hexadec-11-en-1-yl acetate+TX, (Z)-hexadec-13-en-11-yn-1-yl acetate+TX, (Z)-icos-13-en-10-one+TX, (Z)-tetradec-7-en-1-al+TX, (Z)-tetradec-9-en-1-ol+TX, (Z)-tetradec-9-en-1-yl acetate+TX, (7E,9Z)-dodeca-7,9-dien-1-yl acetate+TX, (9Z,11E)-tetradeca-9,11-dien-1-yl acetate+TX, (9Z,12E)-tetradeca-9,12-dien-1-yl acetate+TX, 14-methyloctadec-1-ene+TX, 4-methylnonan-5-ol with 4-methylnonan-5-one+TX, alpha-multistriatin+TX, brevicomin+TX, codlelure+TX, codlemone+TX, cuelure+TX, disparlure+TX, dodec-8-en-1-yl acetate+TX, dodec-9-en-1-yl acetate+TX, dodeca-8+TX, 10-dien-1-yl acetate+TX, dominicalure+TX, ethyl 4-methyloctanoate+TX, eugenol+TX, frontalin+TX, grandlure+TX, grandlure I+TX, grandlure II+TX, grandlure III+TX, grandlure IV+TX, hexalure+TX, ipsdienol+TX, ipsenol+TX, japonilure+TX, lineatin+TX, litlure+TX, looplure+TX, medlure+TX, megatomoic acid+TX, methyl eugenol+TX, muscalure+TX, octadeca-2,13-dien-1-yl acetate+TX, octadeca-3,13-dien-1-yl acetate+TX, orfralure+TX, oryctalure+TX, ostramone+TX, siglure+TX, sordidin+TX, sulcatol+TX, tetradec-11-en-1-yl acetate+TX, trimedlure+TX, trimedlure A+TX, trimedlure Bi+TX, trimedlure B2+TX, trimedlure C+TX, trunc-call+TX, 2-(octylthio)-ethanol+TX, butopyronoxyl+TX, butoxy(polypropylene glycol)+TX, dibutyl adipate+TX, dibutyl phthalate+TX, dibutyl succinate+TX, diethyltoluamide+TX, dimethyl carbate+TX, dimethyl phthalate+TX, ethyl hexanediol+TX, hexamide+TX, methoquin-butyl+TX, methylneodecanamide+TX, oxamate+TX, picaridin+TX, 1-dichloro-1-nitroethane+TX, 1,1-dichloro-2,2-bis(4-ethylphenyl)-ethane+TX, 1,2-dichloropropane with 1,3-dichloropropene+TX, 1-bromo-2-chloroethane+TX, 2,2,2-trichloro-1-(3,4-dichloro-phenyl)ethyl acetate+TX, 2,2-dichlorovinyl 2-ethylsulfinylethyl methyl phosphate+TX, 2-(1,3-dithiolan-2-yl)phenyl dimethylcarbamate+TX, 2-(2-butoxyethoxy)ethyl thiocyanate+TX, 2-(4,5-dimethyl-1,3-dioxolan-2-yl)phenyl methylcarbamate+TX, 2-(4-chloro-3,5-xylyloxy)ethanol+TX, 2-chlorovinyl diethyl phosphate+TX, 2-imidazolidone+TX, 2-isovalerylindan-1,3-dione+TX, 2-methyl(prop-2-ynyl)aminophenyl methylcarbamate+TX, 2-thiocyanatoethyl laurate+TX, 3-bromo-1-chloroprop-1-ene+TX, 3-methyl-1-phenylpyrazol-5-yl dimethyl-carbamate+TX, 4-methyl(prop-2-ynyl)amino-3,5-xylyl methylcarbamate+TX, 5,5-dimethyl-3-oxocyclohex-1-enyl dimethylcarbamate+TX, acethion+TX, acrylonitrile+TX, aldrin+TX, allosamidin+TX, allyxycarb+TX, alpha-ecdysone+TX, aluminium phosphide+TX, aminocarb+TX, anabasine+TX, athidathion+TX, azamethiphos+TX, Bacillus thuringiensis delta endotoxins+TX, barium hexafluorosilicate+TX, barium polysulfide+TX, barthrin+TX, Bayer 22/190+TX, Bayer 22408+TX, beta-cyfluthrin+TX, beta-cypermethrin+TX, bioethanomethrin+TX, biopermethrin+TX, bis(2-chloroethyl) ether+TX, borax+TX, bromfenvinfos+TX, bromo-DDT+TX, bufencarb+TX, butacarb+TX, butathiofos+TX, butonate+TX, calcium arsenate+TX, calcium cyanide+TX, carbon disulfide+TX, carbon tetrachloride+TX, cartap hydrochloride+TX, cevadine+TX, chlorbicyclen+TX, chlordane+TX, chlordecone+TX, chloroform+TX, chloropicrin+TX, chlorphoxim+TX, chlorprazophos+TX, cis-resmethrin+TX, cismethrin+TX, clocythrin+TX, copper acetoarsenite+TX, copper arsenate+TX, copper oleate+TX, coumithoate+TX, cryolite+TX, CS 708+TX, cyanofenphos+TX, cyanophos+TX, cyclethrin+TX, cythioate+TX, d-tetramethrin+TX, DAEP+TX, dazomet+TX, decarbofuran+TX, diamidafos+TX, dicapthon+TX, dichlofenthion+TX, dicresyl+TX, dicyclanil+TX, dieldrin+TX, diethyl 5-methylpyrazol-3-yl phosphate+TX, dilor+TX, dimefluthrin+TX, dimetan+TX, dimethrin+TX, dimethylvinphos+TX, dimetilan+TX, dinoprop+TX, dinosam+TX, dinoseb+TX, diofenolan+TX, dioxabenzofos+TX, dithicrofos+TX, DSP+TX, ecdysterone+TX, El 1642+TX, EMPC+TX, EPBP+TX, etaphos+TX, ethiofencarb+TX, ethyl formate+TX, ethylene dibromide+TX, ethylene dichloride+TX, ethylene oxide+TX, EXD+TX, fenchlorphos+TX, fenethacarb+TX, fenitrothion+TX, fenoxacrim+TX, fenpirithrin+TX, fensulfothion+TX, fenthion-ethyl+TX, flucofuron+TX, fosmethilan+TX, fospirate+TX, fosthietan+TX, furathiocarb+TX, furethrin+TX, guazatine+TX, guazatine acetates+TX, sodium tetrathiocarbonate+TX, halfenprox+TX, HCH+TX, HEOD+TX, heptachlor+TX, heterophos+TX, HHDN+TX, hydrogen cyanide+TX, hyquincarb+TX, IPSP+TX, isazofos+TX, isobenzan+TX, isodrin+TX, isofenphos+TX, isolane+TX, isoprothiolane+TX, isoxathion+TX, juvenile hormone I+TX, juvenile hormone II+TX, juvenile hormone III+TX, kelevan+TX, kinoprene+TX, lead arsenate+TX, leptophos+TX, lirimfos+TX, lythidathion+TX, m-cumenyl methylcarbamate+TX, magnesium phosphide+TX, mazidox+TX, mecarphon+TX, menazon+TX, mercurous chloride+TX, mesulfenfos+TX, metam+TX, metam-potassium+TX, metam-sodium+TX, methanesulfonyl fluoride+TX, methocrotophos+TX, methoprene+TX, methothrin+TX, methoxychlor+TX, methyl isothiocyanate+TX, methylchloroform+TX, methylene chloride+TX, metoxadiazone+TX, mirex+TX, naftalofos+TX, naphthalene+TX, NC-170+TX, nicotine+TX, nicotine sulfate+TX, nithiazine+TX, nornicotine+TX, O-5-dichloro-4-iodophenyl O-ethyl ethylphosphonothioate+TX, O,O-diethyl O-4-methyl-2-oxo-2H-chromen-7-yl phosphorothioate+TX, O,O-diethyl O-6-methyl-2-propylpyrimidin-4-yl phosphorothioate+TX, O,O,O′,O′-tetrapropyl dithiopyrophosphate+TX, oleic acid+TX, para-dichlorobenzene+TX, parathion-methyl+TX, pentachlorophenol+TX, pentachlorophenyl laurate+TX, PH 60-38+TX, phenkapton+TX, phosnichlor+TX, phosphine+TX, phoxim-methyl+TX, pirimetaphos+TX, polychlorodicyclopentadiene isomers+TX, potassium arsenite+TX, potassium thiocyanate+TX, precocene I+TX, precocene II+TX, precocene III+TX, primidophos+TX, profluthrin+TX, promecarb+TX, prothiofos+TX, pyrazophos+TX, pyresmethrin+TX, quassia+TX, quinalphos-methyl+TX, quinothion+TX, rafoxanide+TX, resmethrin+TX, rotenone+TX, kadethrin+TX, ryania+TX, ryanodine+TX, sabadilla)+TX, schradan+TX, sebufos+TX, SI-0009+TX, thiapronil+TX, sodium arsenite+TX, sodium cyanide+TX, sodium fluoride+TX, sodium hexafluorosilicate+TX, sodium pentachlorophenoxide+TX, sodium selenate+TX, sodium thiocyanate+TX, sulcofuron+TX, sulcofuron-sodium+TX, sulfuryl fluoride+TX, sulprofos+TX, tar oils+TX, tazimcarb+TX, TDE+TX, tebupirimfos+TX, temephos+TX, terallethrin+TX, tetrachloroethane+TX, thicrofos+TX, thiocyclam+TX, thiocyclam hydrogen oxalate+TX, thionazin+TX, thiosultap+TX, thiosultap-sodium+TX, tralomethrin+TX, transpermethrin+TX, triazamate+TX, trichlormetaphos-3+TX, trichloronat+TX, trimethacarb+TX, tolprocarb+TX, triclopyricarb+TX, triprene+TX, veratridine+TX, veratrine+TX, XMC+TX, zetamethrin+TX, zinc phosphide+TX, zolaprofos+TX, and meperfluthrin+TX, tetramethylfluthrin+TX, bis(tributyltin) oxide+TX, bromoacetamide+TX, ferric phosphate+TX, niclosamide-olamine+TX, tributyltin oxide+TX, pyrimorph+TX, trifenmorph+TX, 1,2-dibromo-3-chloropropane+TX, 1,3-dichloropropene+TX, 3,4-dichlorotetrahydrothio-phene 1,1-dioxide+TX, 3-(4-chlorophenyl)-5-methylrhodanine+TX, 5-methyl-6-thioxo-1,3,5-thiadiazinan-3-ylacetic acid+TX, 6-isopentenylaminopurine+TX, 2-fluoro-N-(3-methoxyphenyl)-9H-purin-6-amine+TX, benclothiaz+TX, cytokinins+TX, DCIP+TX, furfural+TX, isamidofos+TX, kinetin+TX, Myrothecium verrucaria composition+TX, tetrachlorothiophene+TX, xylenols+TX, zeatin+TX, potassium ethylxanthate+TX, acibenzolar+TX, acibenzolar-S-methyl+TX, Reynoutria sachalinensis extract+TX, alpha-chlorohydrin+TX, antu+TX, barium carbonate+TX, bisthiosemi+TX, brodifacoum+TX, bromadiolone+TX, bromethalin+TX, chlorophacinone+TX, cholecalciferol+TX, coumachlor+TX, coumafuryl+TX, coumatetralyl+TX, crimidine+TX, difenacoum+TX, difethialone+TX, diphacinone+TX, ergocalciferol+TX, flocoumafen+TX, fluoroacetamide+TX, flupropadine+TX, flupropadine hydrochloride+TX, norbormide+TX, phosacetim+TX, phosphorus+TX, pindone+TX, pyrinuron+TX, scilliroside+TX, −sodium fluoroacetate+TX, thallium sulfate+TX, warfarin+TX, −2-(2-butoxyethoxy)ethyl piperonylate+TX, 5-(1,3-benzodioxol-5-yl)-3-hexylcyclohex-2-enone+TX, farnesol with nerolidol+TX, verbutin+TX, MGK 264+TX, piperonyl butoxide+TX, piprotal+TX, propyl isomer+TX, S421+TX, sesamex+TX, sesasmolin+TX, sulfoxide+TX, anthraquinone+TX, copper naphthenate+TX, copper oxychloride+TX, dicyclopentadiene+TX, thiram+TX, zinc naphthenate+TX, ziram+TX, imanin+TX, ribavirin+TX, mercuric oxide+TX, thiophanate-methyl+TX, azaconazole+TX, bitertanol+TX, bromuconazole+TX, cyproconazole+TX, difenoconazole+TX, diniconazole−+TX, epoxiconazole+TX, fenbuconazole+TX, fluquinconazole+TX, flusilazole+TX, flutriafol+TX, furametpyr+TX, hexaconazole+TX, imazalil−+TX, imiben-conazole+TX, ipconazole+TX, metconazole+TX, myclobutanil+TX, paclobutrazole+TX, pefurazoate+TX, penconazole+TX, prothioconazole+TX, pyrifenox+TX, prochloraz+TX, propiconazole+TX, pyrisoxazole+TX, −simeconazole+TX, tebucon-azole+TX, tetraconazole+TX, triadimefon+TX, triadimenol+TX, triflumizole+TX, triticonazole+TX, ancymidol+TX, fenarimol+TX, nuarimol+TX, bupirimate+TX, dimethirimol+TX, ethirimol+TX, dodemorph+TX, fenpropidine+TX, fenpropimorph+TX, spiroxamine+TX, tridemorph+TX, cyprodinil+TX, mepanipyrim+TX, pyrimethanil+TX, fenpiclonil+TX, fludioxonil+TX, benalaxyl+TX, furalaxyl+TX, −metalaxyl−+TX, Rmetalaxyl+TX, ofurace+TX, oxadixyl+TX, carbendazim+TX, debacarb+TX, fuberidazole−+TX, thiabendazole+TX, chlozolinate+TX, dichlozoline+TX, myclozoline−+TX, procymidone+TX, vinclozoline+TX, boscalid+TX, carboxin+TX, fenfuram+TX, flutolanil+TX, mepronil+TX, oxycarboxin+TX, penthiopyrad+TX, thifluzamide+TX, dodine+TX, iminoctadine+TX, azoxystrobin+TX, dimoxystrobin+TX, enestroburin+TX, fenaminstrobin+TX, flufenoxystrobin+TX, fluoxastrobin+TX, kresoxim—methyl+TX, metominostrobin+TX, trifloxystrobin+TX, orysastrobin+TX, picoxystrobin+TX, pyraclostrobin+TX, pyrametostrobin+TX, pyraoxystrobin+TX, ferbam+TX, mancozeb+TX, maneb+TX, metiram+TX, propineb+TX, zineb+TX, captafol+TX, captan+TX, fluoroimide+TX, folpet+TX, tolylfluanid+TX, bordeaux mixture+TX, copper oxide+TX, mancopper+TX, oxine-copper+TX, nitrothal-isopropyl+TX, edifenphos+TX, iprobenphos+TX, phosdiphen+TX, tolclofos-methyl+TX, anilazine+TX, benthiavalicarb+TX, blasticidin-S+TX, chloroneb−+TX, chloro-tha-Ionil+TX, cyflufenamid+TX, cymoxanil+TX, cyclobutrifluram+TX, diclocymet+TX, diclomezine−+TX, dicloran+TX, diethofencarb+TX, dimethomorph−+TX, flumorph+TX, dithianon+TX, ethaboxam+TX, etridiazole+TX, famoxadone+TX, fenamidone+TX, fenoxanil+TX, ferimzone+TX, fluazinam+TX, fluopicolide+TX, flusulfamide+TX, fluxapyroxad+TX, −fenhexamid+TX, fosetyl-aluminium−+TX, hymexazol+TX, iprovalicarb+TX, cyazofamid+TX, methasulfocarb+TX, metrafenone+TX, pencycuron+TX, phthalide+TX, polyoxins+TX, propamocarb+TX, pyribencarb+TX, proquinazid+TX, pyroquilon+TX, pyriofenone+TX, quinoxyfen+TX, quintozene+TX, tiadinil+TX, triazoxide+TX, tricyclazole+TX, triforine+TX, validamycin+TX, valifenalate+TX, zoxamide+TX, mandipropamid+TX, flubeneteram+TX, isopyrazam+TX, sedaxane+TX, benzovindiflupyr+TX, pydiflumetofen+TX, 3-difluoromethyl-1-methyl-1H-pyrazole-4-carboxylic acid (3′,4′,5′-trifluoro-biphenyl-2-yl)-amide+TX, isoflucypram+TX, isotianil+TX, dipymetitrone+TX, 6-ethyl-5,7-dioxo-pyrrolo[4,5][1,4]dithiino[1,2-c]isothiazole-3-carbonitrile+TX, 2-(difluoromethyl)-N-[3-ethyl-1,1-dimethyl-indan-4-yl]pyridine-3-carboxamide+TX, 4-(2,6-difluorophenyl)-6-methyl-5-phenyl-pyridazine-3-carbonitrile+TX, (R)-3-(difluoromethyl)-1-methyl-N-[1,1,3-trimethylindan-4-yl]pyrazole-4-carboxamide+TX, 4-(2-bromo-4-fluoro-phenyl)-N-(2-chloro-6-fluoro-phenyl)-2,5-dimethyl-pyrazol-3-amine+TX, 4-(2-bromo- 4-fluorophenyl)-N-(2-chloro- 6-fluorophenyl)-1, 3-dimethyl-1H-pyrazol-5-amine+TX, fluindapyr+TX, coumethoxystrobin (jiaxiangjunzhi)+TX, Ivbenmixianan+TX, dichlobentiazox+TX, mandestrobin+TX, 3-(4,4-difluoro-3,4-dihydro-3,3-dimethylisoquinolin-1-yl)quinolone+TX, 2-[2-fluoro-6-[(8-fluoro-2-methyl-3-quinolyl)oxy]phenyl]propan-2-ol+TX, oxathiapiprolin+TX, tert-butyl N-[6-[[[(1-methyltetrazol-5-yl)-phenyl-methylene]amino]oxymethyl]-2-pyridyl]carbamate+TX, pyraziflumid+TX, inpyrfluxam+TX, trolprocarb+TX, mefentrifluconazole+TX, ipfentrifluconazole+TX, 2-(difluoromethyl)-N-[(3R)-3-ethyl-1,1-dimethyl-indan-4-yl]pyridine-3-carboxamide+TX, N′-(2,5-dimethyl-4-phenoxy-phenyl)-N-ethyl-N-methyl-formamidine+TX, N′-[4-(4,5-dichlorothiazol-2-yl)oxy-2,5-dimethyl-phenyl]-N-ethyl-N-methyl-formamidine+TX, [2-[3-[2-[1-[2-[3,5-bis(difluoromethyl)pyrazol-1-yl]acetyl]-4-piperidyl]thiazol-4-yl]-4,5-dihydroisoxazol-5-yl]-3-chloro-phenyl] methanesulfonate+TX, but-3-ynyl N-[6-[[(Z)-[(1-methyltetrazol-5-yl)-phenyl-methylene]amino]oxymethyl]-2-pyridyl]carbamate+TX, methyl N-[[5-[4-(2,4-dimethylphenyl)triazol-2-yl]-2-methyl-phenyl]methyl]carbamate+TX, 3-chloro-6-methyl-5-phenyl-4-(2,4,6-trifluorophenyl)pyridazine+TX, pyridachlometyl+TX, 3-(difluoromethyl)-1-methyl-N-[1,1,3-trimethylindan-4-yl]pyrazole-4-carboxamide+TX, 1-[2-[[1-(4-chlorophenyl)pyrazol-3-yl]oxymethyl]-3-methyl-phenyl]-4-methyl-tetrazol-5-one+TX, 1-methyl-4-[3-methyl-2-[[2-methyl-4-(3,4,5-trimethylpyrazol-1-yl)phenoxy]methyl]phenyl]tetrazol-5-one+TX, aminopyrifen+TX, ametoctradin+TX, amisulbrom+TX, penflufen+TX, (Z,2E)-5-[1-(4-chlorophenyl)pyrazol-3-yl]oxy-2-methoxyimino-N,3-dimethyl-pent-3-enamide+TX, florylpicoxamid+TX, fenpicoxamid+TX, tebufloquin+TX, ipflufenoquin+TX, quinofumelin+TX, isofetamid+TX, N-[2-[2,4-dichloro-phenoxy]phenyl]-3-(difluoromethyl)-1-methyl-pyrazole-4-carboxamide TX, N-[2-[2-chloro-4-(trifluoromethyl)phenoxy]phenyl]-3-(difluoromethyl)-1-methyl-pyrazole-4-carboxamide TX, benzothiostrobin+TX, phenamacril+TX, 5-amino-1,3,4-thiadiazole-2-thiol zinc salt (2:1)+TX, fluopyram+TX, flutianil+TX, fluopimomide+TX, pyrapropoyne+TX, picarbutrazox+TX, 2-(difluoromethyl)-N-(3-ethyl-1,1-dimethyl-indan-4-yl)pyridine-3-carboxamide+TX, 2-(difluoromethyl)-N-((3R)-1,1,3-trimethylindan-4-yl) pyridine-3-carboxamide+TX, 4-[[6-[2-(2,4-difluorophenyl)-1,1-difluoro-2-hydroxy-3-(1,2,4-triazol-1-yl)propyl]-3-pyridyl]oxy]benzonitrile+TX, metyltetraprole+TX, 2-(difluoromethyl)-N-((3R)-1, 1, 3-trimethylindan- 4-yl) pyridine-3-carboxamide+TX, α-(1, 1-dimethylethyl)-α-[4″-(trifluoromethoxy) [1, 1″-biphenyl]-4-yl]-5-pyrimidinemethanol+TX, fluoxapiprolin+TX, enoxastrobin+TX, 4-[[6-[2-(2,4-difluorophenyl)-1,1-difluoro-2-hydroxy-3-(1,2,4-triazol-1-yl)propyl]-3-pyridyl]oxy]benzonitrile+TX, 4-[[6-[2-(2,4-difluorophenyl)-1,1-difluoro-2-hydroxy-3-(5-sulfanyl-1,2,4-triazol-1-yl)propyl]-3-pyridyl]oxy] benzonitrile+TX, 4-[[6-[2-(2,4-difluorophenyl)-1,1-difluoro-2-hydroxy-3-(5-thioxo-4H-1,2,4-triazol-1-yl)propyl]-3-pyridyl]oxy]benzonitrile+TX, trinexapac+TX, coumoxystrobin+TX, zhongshengmycin+TX, thiodiazole copper+TX, zinc thiazole+TX, amectotractin+TX, iprodione+TX, N-octyl-N′-[2-(octylamino)ethyl]ethane-1,2-diamine+TX; N′-[5-bromo-2-methyl-6-[(1S)-1-methyl-2-propoxy-ethoxy]-3-pyridyl]-N-ethyl-N-methyl-formamidine+TX, N′-[5-bromo-2-methyl-6-[(1R)-1-methyl-2-propoxy-ethoxy]-3-pyridyl]-N-ethyl-N-methyl-formamidine+TX, N′-[5-bromo-2-methyl-6-(1-methyl-2-propoxy-ethoxy)-3-pyridyl]-N-ethyl-N-methyl-formamidine+TX, N′-[5-chloro-2-methyl-6-(1-methyl-2-propoxy-ethoxy)-3-pyridyl]-N-ethyl-N-methyl-formamidine+TX, N′-[5-bromo-2-methyl-6-(1-methyl-2-propoxy-ethoxy)-3-pyridyl]-N-isopropyl-N-methyl-formamidine+TX (these compounds may be prepared from the methods described in WO2015/155075); N′-[5-bromo-2-methyl-6-(2-propoxypropoxy)-3-pyridyl]-N-ethyl-N-methyl-formamidine+TX (this compound may be prepared from the methods described in IPCOM000249876D); N-isopropyl-N′-[5-methoxy-2-methyl-4-(2,2,2-trifluoro-1-hydroxy-1-phenyl-ethyl)phenyl]-N-methyl-formamidine+TX, N′-[4-(1-cyclopropyl-2,2,2-trifluoro-1-hydroxy-ethyl)-5-methoxy-2-methyl-phenyl]-N-isopropyl-N-methyl-formamidine+TX (these compounds may be prepared from the methods described in WO2018/228896); N-ethyl-N′-[5-methoxy-2-methyl-4-(2-trifluoromethyl)oxetan-2-yl]phenyl]-N-methyl-formamidine+TX, N-ethyl-N′-[5-methoxy-2-methyl-4-[2-trifuoromethyl)tetrahydrofuran-2-yl]phenyl]-N-methyl-formamidine+TX (these compounds may be prepared from the methods described in WO2019/110427); N-[(1R)-1-benzyl-3-chloro-1-methyl-but-3-enyl]-8-fluoro-quinoline-3-carboxamide+TX, N-[(1S)-1-benzyl-3-chloro-1-methyl-but-3-enyl]-8-fluoro-quinoline-3-carboxamide+TX, N-[(1R)-1-benzyl-3,3,3-trifluoro-1-methyl-propyl]-8-fluoro-quinoline-3-carboxamide+TX, N-[(1S)-1-benzyl-3,3,3-trifluoro-1-methyl-propyl]-8-fluoro-quinoline-3-carboxamide+TX, N-[(1R)-1-benzyl-1,3-dimethyl-butyl]-7,8-difluoro-quinoline-3-carboxamide+TX, N-[(1S)-1-benzyl-1,3-dimethyl-butyl]-7,8-difluoro-quinoline-3-carboxamide+TX, 8-fluoro-N-[(1R)-1-[(3-fluorophenyl)methyl]-1,3-dimethyl-butyl]quinoline-3-carboxamide+TX, 8-fluoro-N-[(1S)-1-[(3-fluorophenyl)methyl]-1,3-dimethyl-butyl]quinoline-3-carboxamide+TX, N-[(1R)-1-benzyl-1,3-dimethyl-butyl]-8-fluoro-quinoline-3-carboxamide+TX, N-[(1S)-1-benzyl-1,3-dimethyl-butyl]-8-fluoro-quinoline-3-carboxamide+TX, N-((1R)-1-benzyl-3-chloro-1-methyl-but-3-enyl)-8-fluoro-quinoline-3-carboxamide+TX, N-((1S)-1-benzyl-3-chloro-1-methyl-but-3-enyl)-8-fluoro-quinoline-3-carboxamide+TX (these compounds may be prepared from the methods described in WO2017/153380); 1-(6,7-dimethylpyrazolo[1,5-a]pyridin-3-yl)-4,4,5-trifluoro-3,3-dimethyl-isoquinoline+TX, 1-(6,7-dimethylpyrazolo[1,5-a]pyridin-3-yl)-4,4,6-trifluoro-3,3-dimethyl-isoquinoline+TX, 4,4-difluoro-3,3-dimethyl-1-(6-methylpyrazolo[1,5-a]pyridin-3-yl)isoquinoline+TX, 4,4-difluoro-3,3-dimethyl-1-(7-methylpyrazolo[1,5-a]pyridin-3-yl)isoquinoline+TX, 1-(6-chloro-7-methyl-pyrazolo[1,5-a]pyridin-3-yl)-4,4-difluoro-3,3-dimethyl-isoquinoline+TX (these compounds may be prepared from the methods described in WO2017/025510); 1-(4,5-dimethylbenzimidazol-1-yl)-4,4,5-trifluoro-3,3-dimethyl-isoquinoline+TX, 1-(4,5-dimethylbenzimidazol-1-yl)-4,4-difluoro-3,3-dimethyl-isoquinoline+TX, 6-chloro-4,4-difluoro-3,3-dimethyl-1-(4-methylbenzimidazol-1-yl)isoquinoline+TX, 4,4-difluoro-1-(5-fluoro-4-methyl-benzimidazol-1-yl)-3,3-dimethyl-isoquinoline+TX, 3-(4,4-difluoro-3,3-dimethyl-1-isoquinolyl)-7,8-dihydro-6H-cyclopenta[e]benzimidazole+TX (these compounds may be prepared from the methods described in WO2016/156085); N-methoxy-N-[[4-[5-(trifluoromethyl)-1,2,4-oxadiazol-3-yl]phenyl]methyl]cyclopropanecarboxamide+TX, N,2-dimethoxy-N-[[4-[5-(trifluoromethyl)-1,2,4-oxadiazol-3-yl]phenyl]methyl]propanamide+TX, N-ethyl-2-methyl-N-[[4-[5-(trifluoromethyl)-1,2,4-oxadiazol-3-yl]phenyl]methyl]propanamide+TX, 1-methoxy-3-methyl-1-[[4-[5-(trifluoromethyl)-1,2,4-oxadiazol-3-yl]phenyl]methyl]urea+TX, 1,3-dimethoxy-1-[[4-[5-(trifluoromethyl)-1,2,4-oxadiazol-3-yl]phenyl]methyl]urea+TX, 3-ethyl-1-methoxy-1-[[4-[5-(trifluoromethyl)-1,2,4-oxadiazol-3-yl]phenyl]methyl]urea+TX, N-[[4-[5-(trifluoromethyl)-1,2,4-oxadiazol-3-yl]phenyl]methyl]propanamide+TX, 4,4-dimethyl-2-[[4-[5-(trifluoromethyl)-1,2,4-oxadiazol-3-yl]phenyl]methyl]isoxazolidin-3-one+TX, 5,5-dimethyl-2-[[4-[5-(trifluoromethyl)-1,2,4-oxadiazol-3-yl]phenyl]methyl]isoxazolidin-3-one+TX, ethyl 1-[[4-[5-(trifluoromethyl)-1,2,4-oxadiazol-3-yl]phenyl]methyl]pyrazole-4-carboxylate+TX, N,N-dimethyl-1-[[4-[5-(trifluoromethyl)-1,2,4-oxadiazol-3-yl]phenyl]methyl]-1,2,4-triazol-3-amine+TX. The compounds in this paragraph may be prepared from the methods described in WO 2017/055473, WO 2017/055469, WO 2017/093348 and WO 2017/118689; 2-[6-(4-chlorophenoxy)-2-(trifluoromethyl)-3-pyridyl]-1-(1,2,4-triazol-1-yl)propan-2-ol+TX (this compound may be prepared from the methods described in WO 2017/029179); 2-[6-(4-bromophenoxy)-2-(trifluoromethyl)-3-pyridyl]-1-(1,2,4-triazol-1-yl)propan-2-ol+TX (this compound may be prepared from the methods described in WO 2017/029179); 3-[2-(1-chlorocyclopropyl)-3-(2-fluorophenyl)-2-hydroxy-propyl]imidazole-4-carbonitrile+TX (this compound may be prepared from the methods described in WO 2016/156290); 3-[2-(1-chlorocyclopropyl)-3-(3-chloro-2-fluoro-phenyl)-2-hydroxy-propyl]imidazole-4-carbonitrile+TX (this compound may be prepared from the methods described in WO 2016/156290); (4-phenoxyphenyl)methyl 2-amino-6-methyl-pyridine-3-carboxylate+TX (this compound may be prepared from the methods described in WO 2014/006945); 2,6-Dimethyl-1H,5H-[1,4]dithiino[2,3-c:5,6-c′]dipyrrole-1,3,5,7(2H,6H)-tetrone+TX (this compound may be prepared from the methods described in WO 2011/138281); N-methyl-4-[5-(trifluoromethyl)-1,2,4-oxadiazol-3-yl]benzenecarbothioamide+TX; N-methyl-4-[5-(trifluoromethyl)-1,2,4-oxadiazol-3-yl]benzamide+TX; (Z,2E)-5-[1-(2,4-dichlorophenyl)pyrazol-3-yl]oxy-2-methoxyimino-N,3-dimethyl-pent-3-enamide+TX (this compound may be prepared from the methods described in WO 2018/153707); N′-(2-chloro-5-methyl-4-phenoxy-phenyl)-N-ethyl-N-methyl-formamidine+TX; N′-[2-chloro-4-(2-fluorophenoxy)-5-methyl-phenyl]-N-ethyl-N-methyl-formamidine+TX (this compound may be prepared from the methods described in WO 2016/202742); 2-(difluoromethyl)-N-[(3S)-3-ethyl-1,1-dimethyl-indan-4-yl]pyridine-3-carboxamide+TX (this compound may be prepared from the methods described in WO 2014/095675); (5-methyl-2-pyridyl)-[4-[5-(trifluoromethyl)-1,2,4-oxadiazol-3-yl]phenyl]methanone+TX, (3-methylisoxazol-5-yl)-[4-[5-(trifluoromethyl)-1,2,4-oxadiazol-3-yl]phenyl]methanone+TX (these compounds may be prepared from the methods described in WO 2017/220485); 2-oxo-N-propyl-2-[4-[5-(trifluoromethyl)-1,2,4-oxadiazol-3-yl]phenyl]acetamide+TX (this compound may be prepared from the methods described in WO 2018/065414); ethyl 1-[[5-[5-(trifluoromethyl)-1,2,4-oxadiazol-3-yl]-2-thienyl]methyl]pyrazole-4-carboxylate+TX (this compound may be prepared from the methods described in WO 2018/158365); 2,2-difluoro-N-methyl-2-[4-[5-(trifluoromethyl)-1,2,4-oxadiazol-3-yl]phenyl]acetamide+TX, N-[(E)-methoxyiminomethyl]-4-[5-(trifluoromethyl)-1,2,4-oxadiazol-3-yl]benzamide+TX, N-[(Z)-methoxyiminomethyl]-4-[5-(trifluoromethyl)-1,2,4-oxadiazol-3-yl]benzamide+TX, N-[N-methoxy-C-methyl-carbonimidoyl]-4-[5-(trifluoromethyl)-1,2,4-oxadiazol-3-yl]benzamide+TX (these compounds may be prepared from the methods described in WO 2018/202428); abamectin+TX, acequinocyl+TX, acetamiprid+TX, acetoprole+TX, acrinathrin+TX, acynonapyr+TX, afidopyropen+TX, afoxalaner+TX, alanycarb+TX, allethrin+TX, alpha-cypermethrin+TX, alphamethrin+TX, amidoflumet+TX, aminocarb+TX, azocyclotin+TX, bensultap+TX, benzoximate+TX, benzpyrimoxan+TX, betacyfluthrin+TX, beta-cypermethrin+TX, bifenazate+TX, bifenthrin+TX, binapacryl+TX, bioallethrin+TX, bioallethrin (S)-cyclopentylisomer+TX, bioresmethrin+TX, bistrifluron+TX, broflanilide+TX, brofluthrinate+TX, bromophos-ethyl+TX, buprofezine+TX, butocarboxim+TX, cadusafos+TX, carbaryl+TX, carbosulfan+TX, cartap+TX, CAS number: 1472050-04-6+TX, CAS number: 1632218-00-8+TX, CAS number: 1808115-49-2+TX, CAS number: 2032403-97-5+TX, CAS number: 2044701-44-0+TX, CAS number: 2128706-05-6+TX, CAS number: 2249718-27-0+TX, chlorantraniliprole+TX, chlordane+TX, chlorfenapyr+TX, chloroprallethrin+TX, chromafenozide+TX, clenpirin+TX, cloethocarb+TX, clothianidin+TX, 2-chlorophenyl N-methylcarbamate (CPMC)+TX, cyanofenphos+TX, cyantraniliprole+TX, cyclaniliprole+TX, cycloprothrin+TX, cycloxaprid+TX, cycloxaprid+TX, cyenopyrafen+TX, cyetpyrafen (or etpyrafen)+TX, cyflumetofen+TX, cyfluthrin+TX, cyhalodiamide+TX, cyhalothrin+TX, cypermethrin+TX, cyphenothrin+TX, cyromazine+TX, deltamethrin+TX, diafenthiuron+TX, dialifos+TX, dibrom+TX, dicloromezotiaz+TX, diflovidazine+TX, diflubenzuron+TX, dimpropyridaz+TX, dinactin+TX, dinocap+TX, dinotefuran+TX, dioxabenzofos+TX, emamectin+TX, empenthrin+TX, epsilon-momfluorothrin+TX, epsilon-metofluthrin+TX, esfenvalerate+TX, ethion+TX, ethiprole+TX, etofenprox+TX, etoxazole+TX, famphur+TX, fenazaquin+TX, fenfluthrin+TX, fenitrothion+TX, fenobucarb+TX, fenothiocarb+TX, fenoxycarb+TX, fenpropathrin+TX, fenpyroxymate+TX, fensulfothion+TX, fenthion+TX, fentinacetate+TX, fenvalerate+TX, fipronil+TX, flometoquin+TX, flonicamid+TX, fluacrypyrim+TX, fluazaindolizine+TX, fluazuron+TX, flubendiamide+TX, flubenzimine+TX, flucitrinate+TX, flucycloxuron+TX, flucythrinate+TX, fluensulfone+TX, flufenerim+TX, flufenprox+TX, flufiprole+TX, fluhexafon+TX, flumethrin+TX, fluopyram+TX, flupyradifurone+TX, flupyrimin+TX, fluralaner+TX, fluvalinate+TX, fluxametamide+TX, fosthiazate+TX, gamma-cyhalothrin+TX, Gossyplure™+TX, guadipyr+TX, halofenozide+TX, halofenozide+TX, halofenprox+TX, heptafluthrin+TX, hexythiazox+TX, hydramethylnon+TX, imicyafos+TX, imidacloprid+TX, imiprothrin+TX, indoxacarb+TX, iodomethane+TX, iprodione+TX, isocycloseram+TX, isothioate+TX, ivermectin+TX, kappa-bifenthrin+TX, kappa-tefluthrin+TX, lambda-cyhalothrin+TX, lepimectin+TX, lufenuron+TX, metaflumizone+TX, metaldehyde+TX, metam+TX, methomyl+TX, methoxyfenozide+TX, metofluthrin+TX, metolcarb+TX, mexacarbate+TX, milbemectin+TX, momfluorothrin+TX, niclosamide+TX, nitenpyram+TX, nithiazine+TX, omethoate+TX, oxamyl+TX, oxazosufyl+TX, parathion-ethyl+TX, permethrin+TX, phenothrin+TX, phosphocarb+TX, piperonylbutoxide+TX, pirimicarb+TX, pirimiphos-ethyl+TX, polyhedrosis virus+TX, prallethrin+TX, profenofos+TX, profenofos+TX, profluthrin+TX, propargite+TX, propetamphos+TX, propoxur+TX, prothiophos+TX, protrifenbute+TX, pyflubumide+TX, pymetrozine+TX, pyraclofos+TX, pyrafluprole+TX, pyridaben+TX, pyridalyl+TX, pyrifluquinazon+TX, pyrimidifen+TX, pyrimostrobin+TX, pyriprole+TX, pyriproxyfen+TX, resmethrin+TX, sarolaner+TX, selamectin+TX, silafluofen+TX, spinetoram+TX, spinosad+TX, spirodiclofen+TX, spiromesifen+TX, spiropidion+TX, spirotetramat+TX, sulfoxaflor+TX, tebufenozide+TX, tebufenpyrad+TX, tebupirimiphos+TX, tefluthrin+TX, temephos+TX, tetrachloraniliprole+TX, tetradiphon+TX, tetramethrin+TX, tetramethylfluthrin+TX, tetranactin+TX, tetraniliprole+TX, theta-cypermethrin+TX, thiacloprid+TX, thiamethoxam+TX, thiocyclam+TX, thiodicarb+TX, thiofanox+TX, thiometon+TX, thiosultap+TX, tioxazafen+TX, tolfenpyrad+TX, toxaphene+TX, tralomethrin+TX, transfluthrin+TX, triazamate+TX, triazophos+TX, trichlorfon+TX, trichloronate+TX, trichlorphon+TX, triflumezopyrim+TX, tyclopyrazoflor+TX, zeta-cypermethrin+TX, extract of seaweed and fermentation product derived from melasse+TX, extract of seaweed and fermentation product derived from melasse comprising urea+TX, amino acids+TX, potassium and molybdenum and EDTA-chelated manganese+TX, extract of seaweed and fermented plant products+TX, extract of seaweed and fermented plant products comprising phytohormones+TX, vitamins+TX, EDTA-chelated copper+TX, zinc+TX, and iron+TX, azadirachtin+TX, Bacillus aizawai+TX, Bacillus chitinosporus AQ746 (NRRL Accession No B-21 618)+TX, Bacillus firmus+TX, Bacillus kurstaki+TX, Bacillus mycoides AQ726 (NRRL Accession No. B-21664)+TX, Bacillus pumilus (NRRL Accession No B-30087)+TX, Bacillus pumilus AQ717 (NRRL Accession No. B-21662)+TX, Bacillus sp. AQ178 (ATCC Accession No. 53522)+TX, Bacillus sp. AQ175 (ATCC Accession No. 55608)+TX, Bacillus sp. AQ177 (ATCC Accession No. 55609)+TX, Bacillus subtilis unspecified+TX, Bacillus subtilis AQ153 (ATCC Accession No. 55614)+TX, Bacillus subtilis AQ30002 (NRRL Accession No. B-50421)+TX, Bacillus subtilis AQ30004 (NRRL Accession No. B-50455)+TX, Bacillus subtilis AQ713 (NRRL Accession No. B-21661)+TX, Bacillus subtilis AQ743 (NRRL Accession No. B-21665)+TX, Bacillus thuringiensis AQ52 (NRRL Accession No. B-21619)+TX, Bacillus thuringiensis BD #32 (NRRL Accession No B-21530)+TX, Bacillus thuringiensis subspec. kurstaki BMP 123+TX, Beauveria bassiana+TX, D-limonene+TX, granulovirus+TX, harpin+TX, Helicoverpa armigera Nucleopolyhedrovirus+TX, Helicoverpa zea Nucleopolyhedrovirus+TX, Heliothis virescens Nucleopolyhedrovirus+TX, Heliothis punctigera Nucleopolyhedrovirus+TX, Metarhizium spp.+TX, Muscodor albus 620 (NRRL Accession No. 30547)+TX, Muscodor roseus A3-5 (NRRL Accession No. 30548)+TX, Neem tree based products+TX, Paecilomyces fumosoroseus+TX, Paecilomyces lilacinus+TX, Pasteuria nishizawae+TX, Pasteuria penetrans+TX, Pasteuria ramosa+TX, Pasteuria thornei+TX, Pasteuria usgae+TX, P-cymene+TX, Plutella xylostella Granulosis virus+TX, Plutella xylostella Nucleopolyhedrovirus+TX, Polyhedrosis virus+TX, pyrethrum+TX, QRD 420 (a terpenoid blend)+TX, QRD 452 (a terpenoid blend)+TX, QRD 460 (a terpenoid blend)+TX, Quillaja saponaria+TX, Rhodococcus globerulus AQ719 (NRRL Accession No B-21663)+TX, Spodoptera frugiperda Nucleopolyhedrovirus+TX, Streptomyces galbus (NRRL Accession No. 30232)+TX, Streptomyces sp. (NRRL Accession No. B-30145)+TX, Terpenoid blend+TX, and Verticillium spp.

The compounds of the present invention can be used alone, but are generally formulated into compositions using formulation adjuvants, such as carriers, solvents and surface-active agents (SFAs). Thus, the present invention further provides a composition comprising a compound of the present invention and an agriculturally acceptable formulation adjuvant. There is also provided a composition consisting essentially of a compound of the present invention and an agriculturally acceptable formulation adjuvant. There is also provided a composition consisting of a compound of the present invention and an agriculturally acceptable formulation adjuvant.

The present invention further provides a plant growth regulator composition comprising a compound of the present invention and an agriculturally acceptable formulation adjuvant. There is also provided a plant growth regulator composition consisting essentially of a compound of the present invention and an agriculturally acceptable formulation adjuvant. There is also provided a plant growth regulator composition consisting of a compound of the present invention and an agriculturally acceptable formulation adjuvant.

The present invention further provides a plant abiotic stress management composition comprising a compound of the present invention and an agriculturally acceptable formulation adjuvant. There is also provided a plant abiotic stress management composition consisting essentially of a compound of the present invention and an agriculturally acceptable formulation adjuvant. There is also provided a plant abiotic stress management composition consisting of a compound of the present invention and an agriculturally acceptable formulation adjuvant.

The present invention further provides a seed germination promoter composition comprising a compound of the present invention and an agriculturally acceptable formulation adjuvant. There is also provided a seed germination promoter composition consisting essentially of a compound of the present invention and an agriculturally acceptable formulation adjuvant. There is also provided a seed germination promoter composition consisting of a compound of the present invention and an agriculturally acceptable formulation adjuvant.

The composition can be in the form of concentrates which are diluted prior to use, although ready-to-use compositions can also be made. The final dilution is usually made with water, but can be made instead of, or in addition to, water, with, for example, liquid fertilisers, micronutrients, biological organisms, oil or solvents.

The compositions generally comprise from 0.1 to 99% by weight, especially from 0.1 to 95% by weight, compounds of the present invention are from 1 to 99.9% by weight of a formulation adjuvant which preferably includes from 0 to 25% by weight of a surface-active substance.

The compositions can be chosen from a number of formulation types, many of which are known from the Manual on Development and Use of FAO Specifications for Plant Protection Products, 5th Edition, 1999. These include dustable powders (DP), soluble powders (SP), water soluble granules (SG), water dispersible granules (WG), wettable powders (WP), granules (GR) (slow or fast release), soluble concentrates (SL), oil miscible liquids (OL), ultralow volume liquids (UL), emulsifiable concentrates (EC), dispersible concentrates (DC), emulsions (both oil in water (EW) and water in oil (EO)), micro-emulsions (ME), suspension concentrates (SC), aerosols, capsule suspensions (CS) and seed treatment formulations. The formulation type chosen in any instance will depend upon the particular purpose envisaged and the physical, chemical and biological properties of the compound of the present invention.

Dustable powders (DP) may be prepared by mixing a compound of the present invention with one or more solid diluents (for example natural clays, kaolin, pyrophyllite, bentonite, alumina, montmorillonite, kieselguhr, chalk, diatomaceous earths, calcium phosphates, calcium and magnesium carbonates, sulfur, lime, flours, talc and other organic and inorganic solid carriers) and mechanically grinding the mixture to a fine powder.

Soluble powders (SP) may be prepared by mixing a compound of the present invention with one or more water-soluble inorganic salts (such as sodium bicarbonate, sodium carbonate or magnesium sulphate) or one or more water-soluble organic solids (such as a polysaccharide) and, optionally, one or more wetting agents, one or more dispersing agents or a mixture of said agents to improve water dispersibility/solubility. The mixture is then ground to a fine powder. Similar compositions may also be granulated to form water soluble granules (SG).

Wettable powders (WP) may be prepared by mixing a compound of the present invention with one or more solid diluents or carriers, one or more wetting agents and, preferably, one or more dispersing agents and, optionally, one or more suspending agents to facilitate the dispersion in liquids. The mixture is then ground to a fine powder. Similar compositions may also be granulated to form water dispersible granules (WG).

Granules (GR) may be formed either by granulating a mixture of a compound of the present invention and one or more powdered solid diluents or carriers, or from pre-formed blank granules by absorbing a compound of the present invention (or a solution thereof, in a suitable agent) in a porous granular material (such as pumice, attapulgite clays, fuller's earth, kieselguhr, diatomaceous earths or ground corn cobs) or by adsorbing a compound of the present invention (or a solution thereof, in a suitable agent) on to a hard core material (such as sands, silicates, mineral carbonates, sulphates or phosphates) and drying if necessary. Agents which are commonly used to aid absorption or adsorption include solvents (such as aliphatic and aromatic petroleum solvents, alcohols, ethers, ketones and esters) and sticking agents (such as polyvinyl acetates, polyvinyl alcohols, dextrins, sugars and vegetable oils). One or more other additives may also be included in granules (for example an emulsifying agent, wetting agent or dispersing agent).

Dispersible Concentrates (DC) may be prepared by dissolving a compound of the present invention in water or an organic solvent, such as a ketone, alcohol or glycol ether. These solutions may contain a surface active agent (for example to improve water dilution or prevent crystallisation in a spray tank).

Emulsifiable concentrates (EC) or oil-in-water emulsions (EW) may be prepared by dissolving a compound of the present invention in an organic solvent (optionally containing one or more wetting agents, one or more emulsifying agents or a mixture of said agents). Suitable organic solvents for use in ECs include aromatic hydrocarbons (such as alkylbenzenes or alkylnaphthalenes, exemplified by SOLVESSO 100, SOLVESSO 150 and SOLVESSO 200; SOLVESSO is a Registered Trade Mark), ketones (such as cyclohexanone or methylcyclohexanone) and alcohols (such as benzyl alcohol, furfuryl alcohol or butanol), N-alkylpyrrolidones (such as N-methylpyrrolidone or N-octylpyrrolidone), dimethyl amides of fatty acids (such as C₈-C₁₀ fatty acid dimethylamide) and chlorinated hydrocarbons. An EC product may spontaneously emulsify on addition to water, to produce an emulsion with sufficient stability to allow spray application through appropriate equipment.

Preparation of an EW involves obtaining a compound of the present invention either as a liquid (if it is not a liquid at room temperature, it may be melted at a reasonable temperature, typically below 70° C.) or in solution (by dissolving it in an appropriate solvent) and then emulsifying the resultant liquid or solution into water containing one or more SFAs, under high shear, to produce an emulsion. Suitable solvents for use in EWs include vegetable oils, chlorinated hydrocarbons (such as chlorobenzenes), aromatic solvents (such as alkylbenzenes or alkylnaphthalenes) and other appropriate organic solvents which have a low solubility in water.

Microemulsions (ME) may be prepared by mixing water with a blend of one or more solvents with one or more SFAs, to produce spontaneously a thermodynamically stable isotropic liquid formulation. A compound of the present invention is present initially in either the water or the solvent/SFA blend. Suitable solvents for use in MEs include those hereinbefore described for use in ECs or in EWs. An ME may be either an oil-in-water or a water-in-oil system (which system is present may be determined by conductivity measurements) and may be suitable for mixing water-soluble and oil-soluble pesticides in the same formulation. An ME is suitable for dilution into water, either remaining as a microemulsion or forming a conventional oil-in-water emulsion.

Suspension concentrates (SC) may comprise aqueous or non-aqueous suspensions of finely divided insoluble solid particles of a compound of the present invention. SCs may be prepared by ball or bead milling the solid compound of the present invention in a suitable medium, optionally with one or more dispersing agents, to produce a fine particle suspension of the compound. One or more wetting agents may be included in the composition and a suspending agent may be included to reduce the rate at which the particles settle. Alternatively, a compound of the present invention may be dry milled and added to water, containing agents hereinbefore described, to produce the desired end product.

Aerosol formulations comprise a compound of the present invention and a suitable propellant (for example n-butane). A compound of the present invention may also be dissolved or dispersed in a suitable medium (for example water or a water miscible liquid, such as n-propanol) to provide compositions for use in non-pressurised, hand-actuated spray pumps.

Capsule suspensions (CS) may be prepared in a manner similar to the preparation of EW formulations but with an additional polymerisation stage such that an aqueous dispersion of oil droplets is obtained, in which each oil droplet is encapsulated by a polymeric shell and contains a compound of the present invention and, optionally, a carrier or diluent therefor. The polymeric shell may be produced by either an interfacial polycondensation reaction or by a coacervation procedure. The compositions may provide for controlled release of the compound of the present invention and they may be used for seed treatment. A compound of the present invention may also be formulated in a biodegradable polymeric matrix to provide a slow, controlled release of the compound.

The composition may include one or more additives to improve the biological performance of the composition, for example by improving wetting, retention or distribution on surfaces; resistance to rain on treated surfaces; or uptake or mobility of a compound of the present invention. Such additives include surface active agents (SFAs), spray additives based on oils, for example certain mineral oils or natural plant oils (such as soy bean and rape seed oil), and blends of these with other bio-enhancing adjuvants (ingredients which may aid or modify the action of a compound of the present invention).

Wetting agents, dispersing agents and emulsifying agents may be SFAs of the cationic, anionic, amphoteric or non-ionic type.

Suitable SFAs of the cationic type include quaternary ammonium compounds (for example cetyltrimethyl ammonium bromide), imidazolines and amine salts.

Suitable anionic SFAs include alkali metals salts of fatty acids, salts of aliphatic monoesters of sulphuric acid (for example sodium lauryl sulphate), salts of sulphonated aromatic compounds (for example sodium dodecylbenzenesulphonate, calcium dodecylbenzenesulphonate, butylnaphthalene sulphonate and mixtures of sodium di-isopropyl- and tri-isopropyl-naphthalene sulphonates), ether sulphates, alcohol ether sulphates (for example sodium laureth-3-sulphate), ether carboxylates (for example sodium laureth-3-carboxylate), phosphate esters (products from the reaction between one or more fatty alcohols and phosphoric acid (predominately mono-esters) or phosphorus pentoxide (predominately di-esters), for example the reaction between lauryl alcohol and tetraphosphoric acid; additionally these products may be ethoxylated), sulphosuccinamates, paraffin or olefine sulphonates, taurates and lignosulphonates.

Suitable SFAs of the amphoteric type include betaines, propionates and glycinates.

Suitable SFAs of the non-ionic type include condensation products of alkylene oxides, such as ethylene oxide, propylene oxide, butylene oxide or mixtures thereof, with fatty alcohols (such as oleyl alcohol or cetyl alcohol) or with alkylphenols (such as octylphenol, nonylphenol or octylcresol); partial esters derived from long chain fatty acids or hexitol anhydrides; condensation products of said partial esters with ethylene oxide; block polymers (comprising ethylene oxide and propylene oxide); alkanolamides; simple esters (for example fatty acid polyethylene glycol esters); amine oxides (for example lauryl dimethyl amine oxide); and lecithins.

Suitable suspending agents include hydrophilic colloids (such as polysaccharides, polyvinylpyrrolidone or sodium carboxymethylcellulose) and swelling clays (such as bentonite or attapulgite).

The compound or composition of the present invention may be applied to a plant, part of the plant, plant organ, plant propagation material or a plant growing locus.

The term “plants” refers to all physical parts of a plant, including seeds, seedlings, saplings, roots, tubers, stems, stalks, foliage, and fruits.

The term “locus” as used herein means fields in or on which plants are growing, or where seeds of cultivated plants are sown, or where seed will be placed into the soil. It includes soil, seeds, and seedlings, as well as established vegetation.

The term “plant propagation material” denotes all generative parts of a plant, for example seeds or vegetative parts of plants such as cuttings and tubers. It includes seeds in the strict sense, as well as roots, fruits, tubers, bulbs, rhizomes, and parts of plants.

The application is generally made by spraying the composition, typically by tractor mounted sprayer for large areas, but other methods such as dusting (for powders), drip or drench can also be used. Alternatively, the composition may be applied in furrow or directly to a seed before or at the time of planting.

The compound or composition of the present invention may be applied pre-emergence or post-emergence. Suitably, where the composition is used to regulate the growth of crop plants or enhance the tolerance to abiotic stress, it may be applied post-emergence of the crop. Where the composition is used to promote the germination of seeds, it may be applied pre-emergence.

The present invention envisages application of the compounds or compositions of the invention to plant propagation material prior to, during, or after planting, or any combination of these.

Although active ingredients can be applied to plant propagation material in any physiological state, a common approach is to use seeds in a sufficiently durable state to incur no damage during the treatment process. Typically, seed would have been harvested from the field; removed from the plant; and separated from any cob, stalk, outer husk, and surrounding pulp or other non-seed plant material. Seed would preferably also be biologically stable to the extent that treatment would not cause biological damage to the seed. It is believed that treatment can be applied to seed at any time between seed harvest and sowing of seed including during the sowing process.

Methods for applying or treating active ingredients on to plant propagation material or to the locus of planting are known in the art and include dressing, coating, pelleting and soaking as well as nursery tray application, in furrow application, soil drenching, soil injection, drip irrigation, application through sprinklers or central pivot, or incorporation into soil (broad cast or in band). Alternatively, or in addition active ingredients may be applied on a suitable substrate sown together with the plant propagation material.

The rates of application of compounds of the present invention may vary within wide limits and depend on the nature of the soil, the method of application (pre- or post-emergence; seed dressing; application to the seed furrow; no tillage application etc.), the crop plant, the prevailing climatic conditions, and other factors governed by the method of application, the time of application and the target crop. For foliar or drench application, the compounds of the present invention according to the invention are generally applied at a rate of from 1 to 2000 g/ha, especially from 5 to 1000 g/ha. For seed treatment the rate of application is generally between 0.0005 and 150 g per 100 kg of seed.

The compounds and compositions of the present invention may be applied to dicotyledonous or monocotyledonous crops. Crops of useful plants in which the composition according to the invention can be used include perennial and annual crops, such as berry plants for examples blackberries, blueberries, cranberries, raspberries and strawberries; cereals for example barley, maize (corn), millet, oats, rice, rye, sorghum triticale and wheat; fibre plants for example cotton, hemp, jute and sisal; field crops for example sugar and fodder beet, coffee, hops, mustard, oilseed rape (canola), poppy, sugar cane, sunflower, tea and tobacco; fruit trees for example apple, apricot, avocado, banana, cherry, citrus, nectarine, peach, pear and plum; grasses for example Bermuda grass, bluegrass, bentgrass, centipede grass, fescue, ryegrass, St. Augustine grass and Zoysia grass; herbs such as basil, borage, chives, coriander, lavender, lovage, mint, oregano, parsley, rosemary, sage and thyme; legumes for example beans, lentils, peas and soya beans; nuts for example almond, cashew, ground nut, hazelnut, peanut, pecan, pistachio and walnut; palms for example oil palm; ornamentals for example flowers, shrubs and trees; other trees, for example cacao, coconut, olive and rubber; vegetables for example asparagus, aubergine, broccoli, cabbage, carrot, cucumber, garlic, lettuce, marrow, melon, okra, onion, pepper, potato, pumpkin, rhubarb, spinach and tomato; and vines for example grapes.

Crops are to be understood as being those which are naturally occurring, obtained by conventional methods of breeding, or obtained by genetic engineering. They include crops which contain so-called output traits (e.g. improved storage stability, higher nutritional value and improved flavour).

Crops are to be understood as also including those crops which have been rendered tolerant to herbicides like bromoxynil or classes of herbicides such as ALS-, EPSPS-, GS-, HPPD- and PPO-inhibitors. An example of a crop that has been rendered tolerant to imidazolinones, e.g. imazamox, by conventional methods of breeding is Clearfield® summer canola. Examples of crops that have been rendered tolerant to herbicides by genetic engineering methods include e.g. glyphosate- and glufosinate-resistant maize varieties commercially available under the trade names RoundupReady®, Herculex I® and LibertyLink®.

Crops are also to be understood as being those which naturally are or have been rendered resistant to harmful insects. This includes plants transformed by the use of recombinant DNA techniques, for example, to be capable of synthesising one or more selectively acting toxins, such as are known, for example, from toxin-producing bacteria. Examples of toxins which can be expressed include δ-endotoxins, vegetative insecticidal proteins (Vip), insecticidal proteins of bacteria colonising nematodes, and toxins produced by scorpions, arachnids, wasps and fungi.

An example of a crop that has been modified to express the Bacillus thuringiensis toxin is the Bt maize KnockOut® (Syngenta Seeds). An example of a crop comprising more than one gene that codes for insecticidal resistance and thus expresses more than one toxin is VipCot® (Syngenta Seeds). Crops or seed material thereof can also be resistant to multiple types of pests (so-called stacked transgenic events when created by genetic modification). For example, a plant can have the ability to express an insecticidal protein while at the same time being herbicide tolerant, for example Herculex I® (Dow AgroSciences, Pioneer Hi-Bred International).

Compounds of the present invention may also be used to promote the germination of seeds of non-crop plants, for example as part of an integrated weed control program. A delay in germination of weed seeds may provide a crop seedling with a stronger start by reducing competition with weeds. Alternatively compounds of the present invention may be used to delay the germination of seeds of crop plants, for example to increase the flexibility of timing of planting for the grower.

Normally, in the management of a crop a grower would use one or more other agronomic chemicals or biologicals in addition to the compound or composition of the present invention. There is also provided a mixture comprising a compound or composition of the present invention, and a further active ingredient.

Examples of agronomic chemicals or biologicals include pesticides, such as acaricides, bactericides, fungicides, herbicides, insecticides, nematicides, plant growth regulators, crop enhancing agents, safeners as well as plant nutrients and plant fertilizers. Examples of suitable mixing partners may be found in the Pesticide Manual, 15th edition (published by the British Crop Protection Council). Such mixtures may be applied to a plant, plant propagation material or plant growing locus either simultaneously (for example as a pre-formulated mixture or a tank mix), or sequentially in a suitable timescale. Co-application of pesticides with the present invention has the added benefit of minimising farmer time spent applying products to crops. The combination may also encompass specific plant traits incorporated into the plant using any means, for example conventional breeding or genetic modification.

The present invention provides the use of a compound of Formula (I), or a composition comprising a compound according to Formula (I) and an agriculturally acceptable formulation adjuvant, for improving the tolerance of a plant to abiotic stress, regulating or improving the growth of a plant, promoting seed germination and/or safening a plant against phytotoxic effects of chemicals.

The present invention also provides the use of a compound, composition or mixture of the present invention, for improving the tolerance of a plant to abiotic stress, regulating or improving the growth of a plant, promoting seed germination and/or safening a plant against phytotoxic effects of chemicals.

### Formulation Examples

| Wettable powders | a) | b) | c) |
|---|---|---|---|
| active ingredient [compound of formula (I)] | 25% | 50% | 75% |
| sodium lignosulfonate | 5% | 5% | — |
| sodium lauryl sulfate | 3% | — | 5% |
| sodium diisobutylnaphthalenesulfonate | — | 6% | 10% |
| phenol polyethylene glycol ether | — | 2% | — |
| (7-8 mol of ethylene oxide) | | | |
| highly dispersed silicic acid | 5% | 10% | 10% |
| Kaolin | 62% | 27% | — |

The active ingredient is thoroughly mixed with the adjuvants and the mixture is thoroughly ground in a suitable mill, affording wettable powders that can be diluted with water to give suspensions of the desired concentration.

| Powders for dry seed treatment | a) | b) | c) |
|---|---|---|---|
| active ingredient [compound of formula (I)] | 25% | 50% | 75% |
| light mineral oil | 5% | 5% | 5% |
| highly dispersed silicic acid | 5% | 5% | — |
| Kaolin | 65% | 40% | — |
| Talcum | — | | 20% |

The active ingredient is thoroughly mixed with the adjuvants and the mixture is thoroughly ground in a suitable mill, affording powders that can be used directly for seed treatment.

| Emulsifiable concentrate | |
|---|---|
| active ingredient [compound of formula (I)] | 10% |
| octylphenol polyethylene glycol ether | 3% |
| (4-5 mol of ethylene oxide) | |
| calcium dodecylbenzenesulfonate | 3% |
| castor oil polyglycol ether (35 mol of ethylene oxide) | 4% |
| Cyclohexanone | 30% |
| xylene mixture | 50% |

Emulsions of any required dilution, which can be used in plant protection, can be obtained from this concentrate by dilution with water.

| Dusts | a) | b) | c) |
|---|---|---|---|
| Active ingredient | 5% | 6% | 4% |
| [compound of formula (I)] | | | |
| talcum | 95% | — | — |
| Kaolin | — | 94% | — |
| mineral filler | — | — | 96% |

Ready-for-use dusts are obtained by mixing the active ingredient with the carrier and grinding the mixture in a suitable mill. Such powders can also be used for dry dressings for seed.

| Extruder granules | |
|---|---|
| Active ingredient | 15% |
| [compound of formula (I)] | |
| sodium lignosulfonate | 2% |
| carboxymethylcellulose | 1% |
| Kaolin | 82% |

The active ingredient is mixed and ground with the adjuvants, and the mixture is moistened with water. The mixture is extruded and then dried in a stream of air.

| Coated granules | |
|---|---|
| Active ingredient [compound of formula (I)] | 8% |
| polyethylene glycol (mol. wt. 200) | 3% |
| Kaolin | 89% |

The finely ground active ingredient is uniformly applied, in a mixer, to the kaolin moistened with polyethylene glycol. Non-dusty coated granules are obtained in this manner.

| Suspension concentrate | |
|---|---|
| active ingredient [compound of formula (I)] | 40% |
| propylene glycol | 10% |
| nonylphenol polyethylene glycol ether | 6% |
| (15 mol of ethylene oxide) | |
| Sodium lignosulfonate | 10% |
| carboxymethylcellulose | 1% |
| silicone oil (in the form of a 75% emulsion in water) | 1% |
| Water | 32% |

The finely ground active ingredient is intimately mixed with the adjuvants, giving a suspension concentrate from which suspensions of any desired dilution can be obtained by dilution with water. Using such dilutions, living plants as well as plant propagation material can be treated and protected against infestation by microorganisms, by spraying, pouring or immersion.

| Flowable concentrate for seed treatment | |
|---|---|
| active ingredient [compound of formula (I)] | 40% |
| propylene glycol | 5% |
| copolymer butanol PO/EO | 2% |
| tristyrenephenole with 10-20 moles EO | 2% |
| 1,2-benzisothiazolin-3-one (in the form of a 20% | 0.5% |
| solution in water) | |
| monoazo-pigment calcium salt | 5% |
| Silicone oil (in the form of a 75% emulsion in water) | 0.2% |
| Water | 45.3% |

The finely ground active ingredient is intimately mixed with the adjuvants, giving a suspension concentrate from which suspensions of any desired dilution can be obtained by dilution with water. Using such dilutions, living plants as well as plant propagation material can be treated and protected against infestation by microorganisms, by spraying, pouring or immersion.

Slow Release Capsule Suspension

28 parts of a combination of the compound of formula (I) are mixed with 2 parts of an aromatic solvent and 7 parts of toluene diisocyanate/polymethylene-polyphenylisocyanate-mixture (8:1). This mixture is emulsified in a mixture of 1.2 parts of polyvinyl alcohol, 0.05 parts of a defoamer and 51.6 parts of water until the desired particle size is achieved. To this emulsion a mixture of 2.8 parts 1,6-diaminohexane in 5.3 parts of water is added. The mixture is agitated until the polymerization reaction is completed.

The obtained capsule suspension is stabilized by adding 0.25 parts of a thickener and 3 parts of a dispersing agent. The capsule suspension formulation contains 28% of the active ingredients. The medium capsule diameter is 8-15 microns.

The resulting formulation is applied to seeds as an aqueous suspension in an apparatus suitable for that purpose.

### EXAMPLES

The Examples which follow serve to illustrate the invention.

### Compound Synthesis and Characterisation

The following abbreviations are used throughout this section: s=singlet; bs=broad singlet; d=doublet; dd=double doublet; dt=double triplet; bd=broad doublet; t=triplet; td=triplet doublet; bt=broad triplet; tt=triple triplet; q=quartet; m=multiplet; Me=methyl; Et=ethyl; Pr=propyl; Bu=butyl; DME=1,2-dimethoxyethane; THF=tetrahydrofuran; M.p.=melting point; RT=retention time, MH+=molecular cation (i.e. measured molecular weight).

Throughout this description, temperatures are given in degrees Celsius (° C.) and “m.p.” means melting point. LC/MS means Liquid Chromatography Mass Spectrometry and the description of the apparatus and the following HPLC-MS methods were used for the analysis of the compounds:

Method A: Spectra were recorded on a ZQ Mass Spectrometer from Waters (Single quadrupole mass spectrometer) equipped with an electrospray source (Polarity: positive or negative ions, Capillary: 3.00 kV, Cone: 30.00 V, Extractor: 2.00 V, Source Temperature: 100° C., Desolvation Temperature: 250° C., Cone Gas Flow: 50 L/Hr, Desolvation Gas Flow: 400 L/Hr, Mass range: 100 to 900 Da) and an Acquity UPLC from Waters (Solvent degasser, binary pump, heated column compartment and diode-array detector. Column: Waters UPLC HSS T3, 1.8 μm, 30×2.1 mm, Temp: 60° C., flow rate 0.85 mL/min; DAD Wavelength range (nm): 210 to 500) Solvent Gradient: A=H₂O+5% MeOH+0.05% HCOOH, B=Acetonitrile+0.05% HCOOH) gradient: 0 min 10% B; 0-1.2 min 100% B; 1.2-1.50 min 100% B.

Method B: Spectra were recorded on a ZQ Mass Spectrometer from Waters (Single quadrupole mass spectrometer) equipped with an electrospray source (Polarity: positive or negative ions, Capillary: 3.00 kV, Cone: 30.00 V, Extractor: 2.00 V, Source Temperature: 100° C., Desolvation Temperature: 250° C., Cone Gas Flow: 50 L/Hr, Desolvation Gas Flow: 400 L/Hr, Mass range: 100 to 900 Da) and an Acquity UPLC from Waters (Solvent degasser, binary pump, heated column compartment and diode-array detector. Column: Waters UPLC HSS T3, 1.8 μm, 30×2.1 mm, Temp: 60° C., flow rate 0.85 mL/min; DAD Wavelength range (nm): 210 to 500) Solvent Gradient: A=H₂O+5% MeOH+0.05% HCOOH, B=Acetonitrile+0.05% HCOOH) gradient: 0 min 10% B; 0-2.7 min 100% B; 2.7-3.0 min 100% B.

### Example 1: this Example Illustrates the Preparation of 1,1-dichloro-3,8b-dihydro-2aH-cyclobuta[c]chromen-2-one (Compound IX-a)

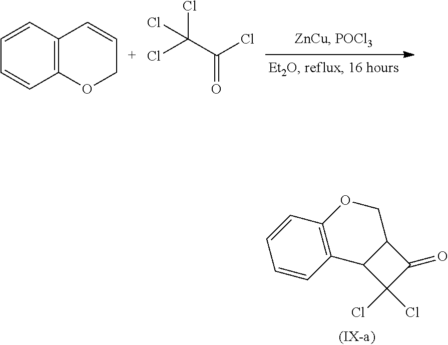

To a flask under argon was added dry diethyl ether (21 mL), 2H-chromene (11.3 mmol, 1.5 g) and cuprouszinc (34.0 mmol, 4.4 g). To this suspension was added a solution of trichloroacetylchloride (22.7 mmol, 2.7 mL) and phosphorus oxychloride (17.0 mmol, 1.6 mL) in diethyl ether (15 mL). After complete addition, the suspension was heated at reflux for 16 hours. The reaction mixture was then filtered through a Celite® pad which was washed with diethyl ether. The filtrate was washed with water, saturated aqueous NaHCO₃ solution and brine. The organic phase was then dried over sodium sulfate, filtered, concentrated under reduced pressure and the obtained crude residue was finally purify by column chromatography on silica gel affording compound of formula (IX-a) as a solid in 67% yield (7.6 mmol, 1.8 g). ¹H NMR (400 MHz, CDCl₃) δ ppm 7.30-7.24 (m, 2H), 7.06 (m, 1H), 6.94 (m, 1H), 4.60 (dd, 1H), 4.32 (m, 1H), 4.23 (d, 1H), 3.87 (dd, 1H).

### Example 2: this Example Illustrates the Preparation of 1,1-dichloro-3,3-dimethyl-2a,8b-dihydrocyclobuta[c]chromen-2-one (Compound IX-b)

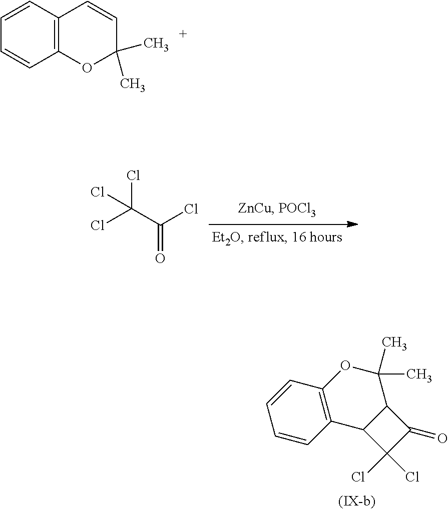 [SMILES: CC1(C)C=Cc2ccccc2O1 ; O=C(Cl)C(Cl)(Cl)Cl ; CC1(C)Oc2ccccc2C2C1C(=O)C2(Cl)Cl]

To a flask under argon was added dry diethyl ether (21 mL), 2H-chromene (11.3 mmol, 1.5 g) and cuprouszinc (34.0 mmol, 4.4 g). To this suspension was added a solution of trichloroacetylchloride (22.7 mmol, 2.7 mL) and phosphorus oxychloride (17.0 mmol, 1.6 mL) in diethyl ether (15 mL). After complete addition, the suspension was heated at reflux for 16 hours. The reaction mixture was then filtered through a Celite® pad which was washed with diethyl ether. The filtrate was washed with water, saturated aqueous NaHCO₃ solution and brine. The organic phase was then dried over sodium sulfate, filtered, concentrated under reduced pressure and the obtained crude residue was finally purify by column chromatography on silica gel affording compound of formula (IX-b) as a solid in 67% yield (7.6 mmol, 1.8 g). ¹H NMR (400 MHz, CDCl₃) δ ppm 7.28-7.22 (m, 2H), 7.02 (m, 1H), 6.87 (m, 1H), 4.17 (d, 1H), 4.10 (d, 1H), 1.70 (s, 3H), 1.18 (s, 3H).

### Example 3: this Example Illustrates the Preparation of 1,3a,4,9b-tetrahydrofuro[2,3-c]chromen-2-one (Compound IV-a)

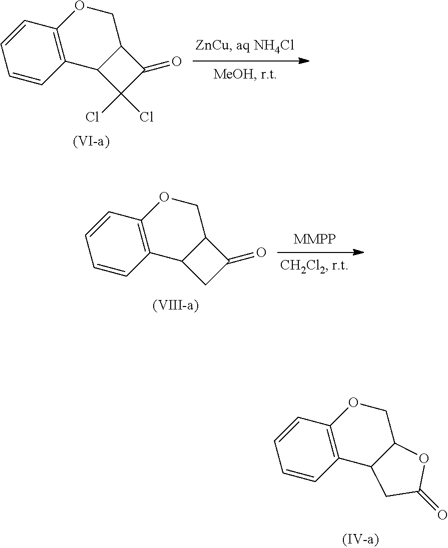 [SMILES: O=C1C2COc3ccccc3C2C1(Cl)Cl ; O=C1CC2c3ccccc3OCC12 ; O=C1CC2c3ccccc3OCC2O1]

Compound of formula (VI-a, 5.35 mmol, 1.3 g) was dissolved in a saturated solution of ammonium chloride (0.57 g) in methanol (36 mL). Cuprouszinc (1.03 g) was added and the resulting suspension was stirred at room temperature for 16 hours. The reaction mixture was then filtered through a Celite® pad which was washed with EtOAc. HCl aqueous solution (1M) was added to the organic solution, the phases separated and the organic layer was dried over Na₂SO₄, filtered and concentrated under reduced pressure to afford compound of formula (VIII-a) as an oil in quantitative yield (0.98 g). ¹H NMR (400 MHz, CDCl₃) δ ppm 7.24-7.16 (m, 2H), 7.01 (td, 1H), 6.96 (m, 1H), 4.50 (m, 1H), 3.90-3.82 (m, 2H), 3.80-3.73 (m, 1H), 3.70-3.59 (m, 1H), 2.88 (m, 1H). Compound of formula (XI-a, 5.17 mmol, 0.9 g) was then dissolved in methanol (22 mL) and water (11.4 mL), magnesium monoperoxyphthalate (MMPP) was added in one portion and the reaction mixture was heated to 40° C. After 12 hours, the reaction was cooled to room temperature, quenched with a 10% aqueous Na₂S₂O₃ and the organic phase washed with brine, dried over sodium sulfate and concentrated under reduced pressure affording compound of formula (IV-a) in 55% yield (0.74 g). ¹H NMR (400 MHz, CDCl₃) δ ppm 7.18 (m, 1H), 7.10 (m, 1H), 7.00 (m, 1H), 6.91 (m, 1H), 4.93 (m, 1H), 4.37 (dd, 1H), 4.07 (dd, 1H), 3.84 (m, 1H), 3.15 (dd, 1H), 2.62 (dd, 1H).

### Example 4: this Example Illustrates the Preparation of 4,4-dimethyl-3a,9b-dihydro-1H-furo[2,3-c]chromen-2-one (Compound IV-b)

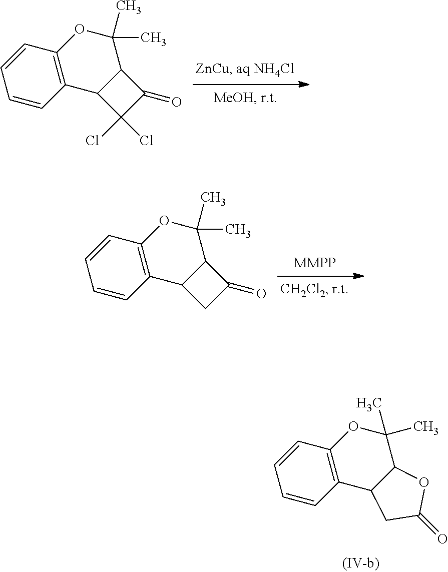 [SMILES: CC1(C)Oc2ccccc2C2C1C(=O)C2(Cl)Cl ; CC1(C)Oc2ccccc2C2CC(=O)C21 ; CC1(C)Oc2ccccc2C2CC(=O)OC21]

Compound of formula (VI-a, 5.35 mmol, 1.3 g) was dissolved in a saturated solution of ammonium chloride (0.57 g) in methanol (36 mL). Cuprouszinc (1.03 g) was added and the resulting suspension was stirred at room temperature for 16 hours. The reaction mixture was then filtered through a Celite® which was washed with EtOAc. HCl aqueous solution (1M) was added to the organic solution, the phases separated and the organic layer was dried over Na₂SO₄, filtered and concentrated under reduced pressure to afford compound of formula (VIII-a) as an oil in quantitative yield (0.98 g). ¹H NMR (400 MHz, CDCl₃) δ ppm 7.24-7.16 (m, 2H), 7.01 (td, 1H), 6.96 (m, 1H), 4.50 (m, 1H), 3.90-3.82 (m, 2H), 3.80-3.73 (m, 1H), 3.70-3.59 (m, 1H), 2.88 (m, 1H). Compound of formula (XI-a, 5.17 mmol, 0.9 g) was then dissolved in methanol (22 mL) and water (11.4 mL), MMPP was added in one portion and the reaction mixture was heated to 40° C. After 12 hours, the reaction was cooled to room temperature, quenched with a 10% aqueous Na₂S₂O₃ and the organic phase washed with brine, dried over sodium sulfate and concentrated under reduced pressure affording compound of formula (IV-b) in 55% yield (0.74 g). ¹H NMR (400 MHz, CDCl₃) δ ppm 7.16 (m 1H), 7.08 (m, 1H), 6.95 (m, 1H), 6.84 (m, 1H), 4.49 (d, 1H), 3.76 (m, 1H), 3.13 (dd, 1H), 2.70 (dd, 1H), 1.56 (s, 3H), 1.26 (s, 3H).

### Example 5: this Example Illustrates the Preparation of 1,3,3a,8b-tetrahydrobenzofuro[2,3-b]pyrrol-2-one (Compound IV-c)

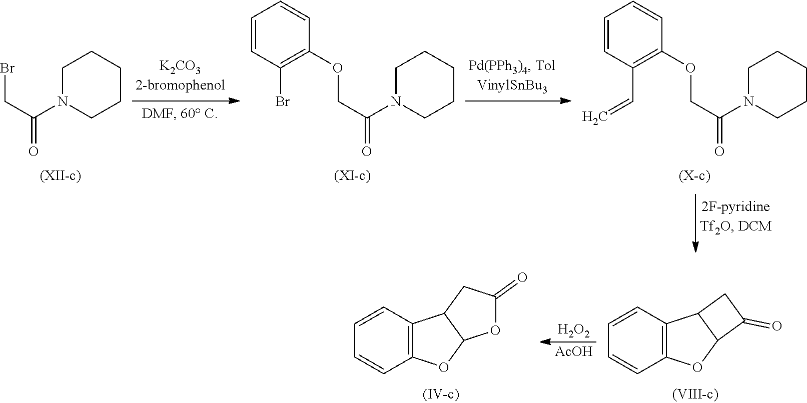

To a solution of compound (XII-c, 10.5 g, 51 mmol) and K₂CO₃ (2.0 eq, 102 mmol) in DMF (100 mL) was added at 0° C. 2-bromophenol (1.2 equiv., 61 mmol). The reaction was then heated under an argon atmosphere at 60° C. for 90 minutes. The reaction mixture was then partitioned between water and CH₂Cl₂ and the phases separated. The aqueous phase was extracted with a further portion of CH₂Cl₂ and the combined organic layers were washed with water, dried over magnesium sulphate and concentrated under vacuum. The resulting crude residue was purified by flash chromatography on SiO₂ affording compound (XI-c) as a colorless oil that crystallized on standing in 92% yield (14.0 g). LCMS (Method A): RT 0.95 min; ES⁺ 300 (M+H⁺).

A solution compound (XI-c) (16.5 g, 3.35 mmol) in toluene (220 mL) was degassed with argon for 30 minutes, and vinyl stannane (1.3 equiv., 72 mmol) follow by Pd(PPh₃)₄ (2.8 mmol, 99.8 mass %) were added. The resulting reaction mixture was heated to 100° C. and stirred for 16 hours. The reaction mixture was then cooled, concentrated under vacuum and purified by flash chromatography on SiO₂ affording compound (X-c) as a colourless oil in 95% yield (12.9 g, 52.6 mmol). LCMS (Method A): RT 0.97 min; ES⁺ 247 (M+H⁺).

To a stirred solution of compound (X-c, 11 g, 44.8 mmol) in CH₂Cl₂ (179 mL) was added sequentially at room temperature 2-fluoropyridine (1.3 eq, 58.3 mmol, 5.1 mL) and Tf₂O (1.2 eq, 53.8 mmol, 9.05 mL) dropwise and the resulting reaction mixture was stirred for 14 hours. Water (100 mL) was then added to the reaction and the biphasic mixture was stirred at room temperature for additional 16 hours. The reaction mixture was extracted with CH₂Cl₂, dried over sodium sulfate and concentrated under reduced pressure. Purification by flash chromatography on SiO₂ afforded compound (VIII-c) in 77% yield (5.5 g, 34 mmol). ¹H NMR (400 MHz, CDCl₃) δ ppm 7.32 (m, 1H), 7.22 (m, 1H), 6.98 (td, 1H), 6.91 (m, 1H), 5.75 (dt, 1H), 4.25 (td, 1H), 3.67 (ddd, 1H), 3.11 (dt, 1H).

To a solution of compound (VIII-c) (580 mg, 3.62 mmol) in AcOH (12 mL) at room temperature was added a 30% H₂O₂ solution (5 eq, 1.85 mL). The reaction was stirred for 16 hours at room temperature and then quenched with a saturated solution of NaHCO₃ (100 mL) and EtOAc (100 mL). The organic layer was then dried over sodium sulfate and concentrated under vacuum. Purification by flash chromatography on SiO₂ afforded compound (IV-c) as a colourless oil, which crystallized on standing in 80% yield (511 g, 2.90 mmol). LCMS (Method A): RT 0.70 min; ES⁺ 177 (M+H⁺).

Using a similar procedure to Example 5, the following compounds were prepared:

### Compound (IV-d): 3a,8b-dihydro-1H-benzothiopheno[2,3-b]furan-2-one

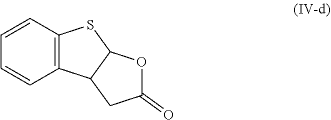 [SMILES: O=C1CC2c3ccccc3SC2O1]

LCMS (Method A): RT 0.77 min; ES⁺ 193 (M+H⁺).

### Compound (IV-e): 4-methylsulfonyl-3a,8b-dihydro-1H-furo[2,3-b]indol-2-one

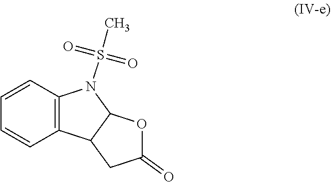 [SMILES: CS(=O)(=O)N1c2ccccc2C2CC(=O)OC21]

LCMS (Method A): RT 0.68 min; ES⁺ 254 (M+H⁺).

### Example 6: this Example Illustrates the Preparation of (1Z)-1-(hydroxymethylene)-3a,8b-dihydrofuro[2,3-b]benzofuran-2-one (Compound II-c)

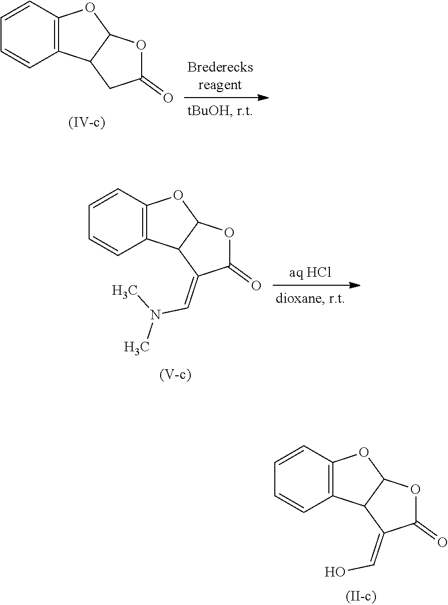 [SMILES: O=C1CC2c3ccccc3OC2O1 ; CN(C)/C=C1/C(=O)OC2Oc3ccccc3C12 ; O=C1OC2Oc3ccccc3C2/C1=C\O]

Compound of formula (IV-c) (5.7 mmol, 1.0 g) was suspended in dry toluene (or tert-butanol) and Bredereck's reagent (tert butoxybis(dimethylamino)methane) was then added (19.9 mmol, 3.5 g) under argon and the reaction mixture was stirred at room temperature for 2 hours. The reaction mixture was diluted with ethyl acetate (50 mL) and washed with water followed by brine, dried over Na₂SO₄ and the solvent evaporated under reduced pressure affording compound (V-c). Compound of formula (V-c) (5.2 mmol, 1.2 g) was then dissolved in 1,4-dioxane (10 mL) and aqueous hydrochloric acid solution (2M, 5.2 mL) and the resulting reaction mixture was stirred for 35 minutes at room temperature. Brine was added and extraction was done with ethyl acetate. The combined organic fractions were dried over sodium sulfate, the solvents evaporated and the resulting crude was used without further purification. Compound of formula (II-c) in 81% yield (4.5 mmol, 0.92 g). LCMS (Method A): RT 0.72 min; ES⁺ 203 (M−H⁺).

TABLE 2
LC/MS data (Rt = Retention time) for selected compounds of formula (II), which were prepared
using the procedure of Example 6.

| | Compound | | |
|---|---|---|---|
| No. | Name | Structure | LC/MS |
| II-d | 1-(hydroxy methylene)-3a,8b- dihydrobenzothiopheno [2,3-b]furan-2-one | 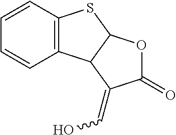 [SMILES: O=C1OC2Sc3ccccc3C2C1=CO] | RT 0.78 min; ES⁻ 219 (M − H⁺) |
| II-e | 1-(hydroxy methylene)-4- methylsulfonyl-3a,8b- dihydrofuro[2,3- b]indol-2-one | 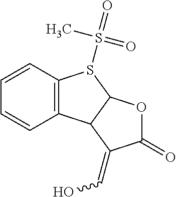 | RT 0.69 min; ES⁻ 280 (M − H⁺) |
| II-a | 1-(hydroxy methylene)-4,9b- dihydro-3aH-furo[2,3- c]chromen-2-one | 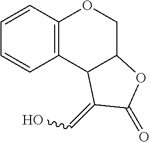 [SMILES: O=C1OC2COc3ccccc3C2C1=CO] | RT 0.68 min; ES⁻ 217 (M − H⁺) |
| II-b | 1-(hydroxy methylene)-4,4- dimethyl-3a,9b- dihydrofuro[2,3- c]chromen-2-one | 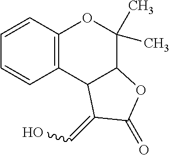 [SMILES: CC1(C)Oc2ccccc2C2C(=CO)C(=O)OC21] | RT 0.84 min; ES⁻ 245 (M − H⁺) |

### Example 7: This example illustrates the preparation of tert-butyl (1E)-1-[(4-methyl-5-oxo-2H-furan-2-yl)oxymethylene]-2-oxo-4,8b-dihydro-3aH-indeno[2,1-b]pyrrole-3-carboxylate (Compound I-1)

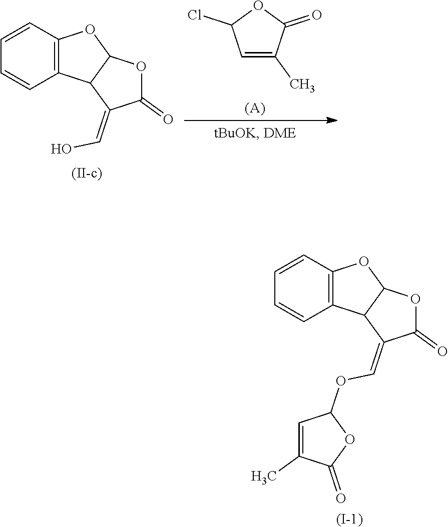

Compound of formula (II-c) (1.32 mmol) was dissolved in anhydrous 1,2-dimethoxyethane (4 mL), the resulting solution cooled to 0° C. and tBuOK (0.19 g, 1.72 mmol) was then added. After 10 minutes at 0° C., known compound of formula (A) (0.74 mmol) was added as a solution in DME (1 mL). The reaction mixture was then slowly warmed to room temperature. After 16 hours, a saturated aqueous NH₄Cl solution was added and the reaction mixture was extracted with ethyl acetate. The combined organic extracts were washed with brine, dried over sodium sulfate and concentrated under vacuum.

The crude reaction residue was purified by flash chromatography on silica gel affording compound of formula (I-1) as a colorless foam and as a mixture of diastereoisomers in 72% yield (0.95 mmol). ¹H NMR (400 MHz, CDCl₃) δ ppm (data given for the two diastereoisomers) 7.61 (d, 0.5H), 7.60 (d, 0.5 H), 7.21-7.33 (m, 2H), 7.06-7.09 (m, 0.5H), 7.03-7.06 (m, 0.5H), 6.96-7.01 (m, 1H), 6.91-6.96, (m, 1H), 6.54 (d, 0.5H), 6.53 (d, 0.5H), 6.27-6.31 (m, 1H), 4.85 (d, 1H), 2.09 (m, 3H).

### Example 8: This example illustrates the preparation of (1E)-1-[(4-methyl-5-oxo-2H-furan-2-yl)oxymethylene]-4-oxo-3a,8b-dihydrobenzothiopheno[2,3-b]furan-2-one and (1E)-1-[(4-methyl-5-oxo-2H-furan-2-yl)oxymethylene]-4,4-dioxo-3a,8b-dihydrobenzothiopheno[2,3-b]furan-2-one (Compounds I-13 and I-19)

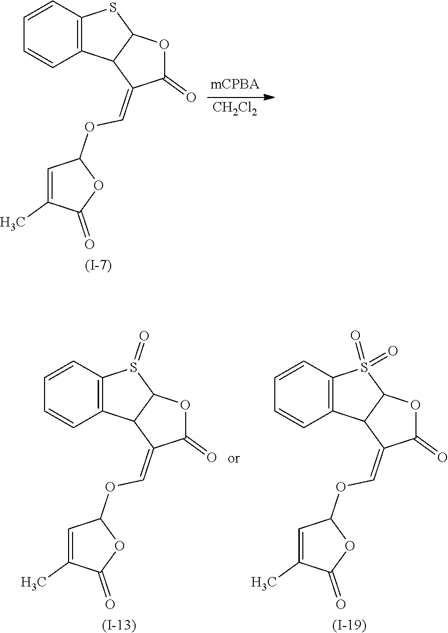

Compound of formula (I-7) (60 mg, 0.19 mmol) was dissolved in dichloromethane (2 mL), the resulting solution cooled to 0° C. and mCPBA (77 mass %, 0.042 g, 0.19 mmol) was then added. The reaction was stirred for 10 minutes at 0° C. Dichloromethane (10 mL) and a saturated aqueous NaHCO₃ solution were successively added and the organic phase was washed with a saturated NaHCO₃ aqueous solution. The organic phase was dried over sodium sulfate and concentrated under vacuum. The crude reaction residue was purified by flash chromatography on silica gel affording compound of formula (I-13) as a colorless foam. The product is obtained as a mixture of only 2 diastereoisomers in 75% yield (47 mg, 0.14 mmol). ¹H NMR (400 MHz, CDCl₃) δ ppm (data given for the two diastereoisomers) 7.92-7.87 (m, 1H), 7.70-7.54 (m, 4H), 7.10-7.05 (m, 1H), 6.35 (br s, 0.5H), 6.32 (br s, 0.5H), 5.73 (two d, J=7.3 Hz, 1H), 5.51 (br d, J=5.9 Hz, 1H), 2.14-2.10 (m, 3H).

Using the same procedure, but with 2.5 equivalents of mCPBA (77 mass %, 105 mg, 0.48 mmol), compound of formula (I-19) was obtained as a colorless foam in 70% yield (46 mg, 0.13 mmol). ¹H NMR (400 MHz, CD₃CN) δ ppm (data given for the two diastereoisomers) 7.83-7.60 (m, 5H), 7.17 (m, 0.5H), 7.14 (m, 0.5H), 6.42-6.40 (m, 0.5H), 6.40-6.38 (m, 0.5H), 5.73 (d, 0.5H), 5.72 (d, 0.5H), 5.17-5.15 (m, 0.5H), 5.15-5.14 (m, 0.5H), 2.01-1.99 (m, 3H).

TABLE 3
LC/MS data (Rₜ = Retention time) for selected compounds of formula (I), which were prepared
using the procedures of Example 7 or Example 8.

| | Compound | | |
|---|---|---|---|
| No. | Name | Structure | LC/MS |
| I-1 | (1E)-1-[(4-methyl-5- oxo-2H-furan-2- yl)oxymethylene]- 3a,8b-dihydrofuro[2,3- b]benzofuran-2-one | 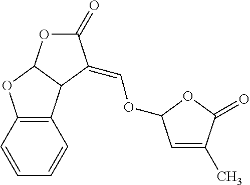 [SMILES: CC1=CC(O/C=C2/C(=O)OC3Oc4ccccc4C23)OC1=O] | Rₜ = 0.86 and 0.88 min; ES+ 301 (M + H⁺) |
| I-2 | (1E)-1-[(3,4-dimethyl- 5-oxo-2H-furan-2- yl)oxymethylene]- 3a,8b-dihydrofuro[2,3- b]benzofuran-2-one | 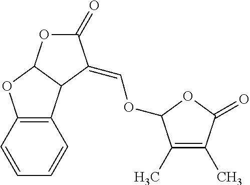 [SMILES: CC1=C(C)C(O/C=C2/C(=O)OC3Oc4ccccc4C23)OC1=O] | Rₜ = 0.90 and 0.91 min; ES+ 315 (M + H⁺) |
| I-7 | (1E)-1-[(4-methyl-5- oxo-2H-furan-2- yl)oxymethylene]- 3a,8b- dihydrobenzothiopheno [2,3-b]furan-2-one | 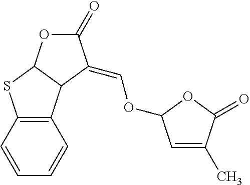 [SMILES: CC1=CC(O/C=C2/C(=O)OC3Sc4ccccc4C23)OC1=O] | Rₜ = 0.90 and 0.92 min; ES+ 317 (M + H⁺) |
| I-8 | (1E)-1-[(3,4-dimethyl- 5-oxo-2H-furan-2- yl)oxymethylene]- 3a,8b- dihydrobenzothiopheno [2,3-b]furan-2-one | 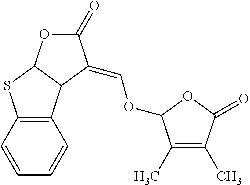 [SMILES: CC1=C(C)C(O/C=C2/C(=O)OC3Sc4ccccc4C23)OC1=O] | Rₜ = 0.94 and 0.96 min; ES+ 331 (M + H⁺) |
| I-13 | (1E)-1-[(4-methyl-5- oxo-2H-furan-2- yl)oxymethylene]-4- oxo-3a,8b- dihydrobenzothiopheno [2,3-b]furan-2-one | 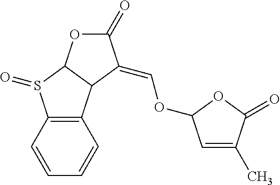 [SMILES: CC1=CC(O/C=C2/C(=O)OC3C2c2ccccc2S3=O)OC1=O] | Rₜ = 0.71 min; ES+ 333 (M + H⁺) |
| I-14 | (1E)-1-[(3,4-dimethyl- 5-oxo-2H-furan-2- yl)oxymethylene]- 3a,8b-dihydrofuro[2,3- b]benzofuran-2-one | 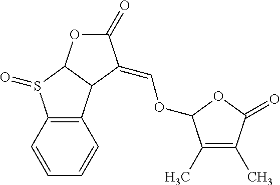 [SMILES: CC1=C(C)C(O/C=C2/C(=O)OC3C2c2ccccc2S3=O)OC1=O] | Rₜ = 0.73 min; ES+ 347 (M + H⁺) |
| I-19 | (1E)-1-[(4-methyl-5- oxo-2H-furan-2- yl)oxymethylene]-4,4- dioxo-3a,8b- dihydrobenzothiopheno [2,3-b]furan-2-one | 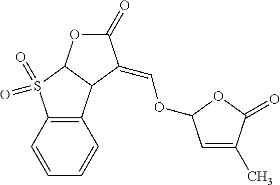 [SMILES: CC1=CC(O/C=C2/C(=O)OC3C2c2ccccc2S3(=O)=O)OC1=O] | Rₜ = 0.71 min; ES+ 349 (M + H⁺) |
| I-20 | (1E)-1-[(3,4-dimethyl- 5-oxo-2H-furan-2- yl)oxymethylene]-4,4- dioxo-3a,8b- dihydrobenzothiopheno [2,3-b]furan-2-one | 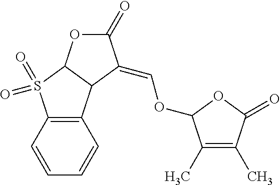 [SMILES: CC1=C(C)C(O/C=C2/C(=O)OC3C2c2ccccc2S3(=O)=O)OC1=O] | Rₜ = 0.78 min; ES+ 363 (M + H⁺) |
| I-25 | (1E)-1-[(4-methyl-5- oxo-2H-furan-2- yl)oxymethylene]-4- methylsulfonyl-3a,8b- dihydrofuro[2,3- b]indol-2-one | 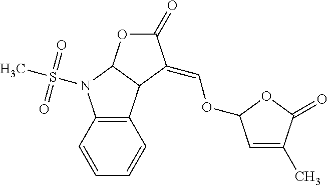 [SMILES: CC1=CC(O/C=C2/C(=O)OC3C2c2ccccc2N3S(C)(=O)=O)OC1=O] | Rₜ = 0.84 min; ES+ 378 (M + H⁺) |
| I-26 | (1E)-1-[(3,4-dimethyl- 5-oxo-2H-furan-2- yl)oxymethylene]-4- methylsulfonyl-3a,8b- dihydrofuro[2,3- b]indol-2-one | 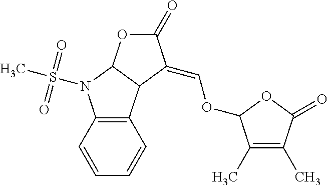 [SMILES: CC1=C(C)C(O/C=C2/C(=O)OC3C2c2ccccc2N3S(C)(=O)=O)OC1=O] | Rₜ = 0.87 min; ES+ 392 (M + H⁺) |
| I-31 | (1E)-1-[(4-methyl-5- oxo-2H-furan-2- yl)oxymethylene]- 4,9b-dihydro-3aH- furo[2,3-c]chromen-2- one | 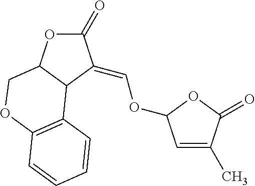 [SMILES: CC1=CC(O/C=C2/C(=O)OC3COc4ccccc4C23)OC1=O] | Rₜ = 0.82 min (A); MS: m/z = 315 (M + 1) |
| I-37 | (1E)-4,4-dimethyl-1- [(4-methyl-5-oxo-2H- furan-2- yl)oxymethylene]- 3a,9b-dihydrofuro[2,3- c]chromen-2-one | 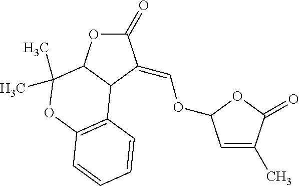 [SMILES: CC1=CC(O/C=C2/C(=O)OC3C2c2ccccc2OC3(C)C)OC1=O] | Rₜ = 0.95 min (A); MS: m/z = 343 (M + 1) |

### Biological Examples

### Example B1: Dark Induced Senescence of Corn Leaf

It is known that strigolactones regulate (accelerate) leaf senescence, potentially through D14 receptor signaling.

Corn plants of variety Multitop were grown in a greenhouse with relative 75% humidity and at 23-25° C. for 6 weeks. 1.4 cm diameter leaf discs were placed into 24-well plates containing test compounds in a concentration gradient (100 μM-0.0001 μM) at a final concentration of 0.5% DMSO. Each concentration was tested in 12 replicates. Plates were sealed with seal foil. The foil was pierced to provide gas exchange in each well. The plates were placed into the completely dark climatic chamber. Plates were incubated in the chamber with 75% humidity and at 23° C. for 8 days. On days 0, 5, 6, 7 and 8 photographs were taken of each plate, and image analysis conducted with a macro developed using the ImageJ software. The image analysis was used to determine the concentration at which 50% senescence was achieved (IC50), see Table 4. The lower the value, the higher senescence induction potency.

TABLE 4
IC50 of compounds (I) for dark
induced senescence of corn leaf

| Compound | IC50 (μM) |
|---|---|
| I-1 | 0.03 |
| I-2 | 0.015 |
| I-8 | 0.04 |
| I-13 | 0.015 |
| I-31 | 0.03 |

1. A compound of formula (I): 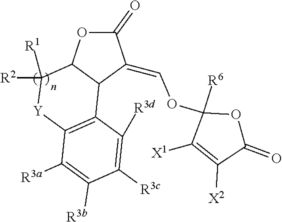
wherein
Y is O, N—R⁴, S, S(O), or S(O)₂;
n is 0 or 1;
R¹ and R² are each independently selected from hydrogen and C₁-C₄alkyl; or
R¹ and R² together with the carbon atom to which they are attached form a C₃-C₆cycloalkyl group;
R^{3a}, R^{3b}, R^{3c}, R^{3d} are each independently selected from hydrogen, halogen, cyano, C₁-C₄alkyl, C₁-C₄haloalkyl, C₁-C₄alkoxy, C₁-C₄alkoxycarbonyl, C₁-C₄alkylcarbonyl, and C₃-C₆cycloalkyl, wherein each cycloalkyl moiety is optionally substituted with 1 to 3 groups represented by R⁵;
R⁴ is hydrogen, C₁-C₄alkyl, formyl, C₁-C₄alkylcarbonyl, C₁-C₄alkoxycarbonyl, C₁-C₄haloalkylcarbonyl, C₃-C₈cycloalkylcarbonyl, phenyl, —S(O)₂—C₁-C₄alkyl, or —S(O)₂-phenyl;
R⁵ is halogen, cyano, C₁-C₄alkyl, C₁-C₄haloalkyl, or C₁-C₄alkoxy;
R⁶ is hydrogen or C₁-C₄alkyl; and
X¹ and X² are each independently selected from hydrogen, halogen, cyano, C₁-C₄alkyl, and C₁-C₄alkoxy;
or a salt or an N-oxide thereof.

2. The compound according to claim 1, wherein R¹ and R² are each independently selected from hydrogen and methyl.

3. The compound according to claim 1, wherein R^{3a}, R^{3b}, R^{3c}, R^{3d} are all hydrogen.

4. The compound according to claim 1, wherein R⁶ is hydrogen.

5. The compound according to claim 1, wherein R⁴ is —S(O)₂-C₁-C₄alkyl.

6. The compound according to claim 1, wherein R⁴ is —S(O)₂methyl.

7. The compound according to claim 1, wherein X¹ and X² are each independently selected from hydrogen, halogen, C₁-C₃alkyl, and C₁-C₃alkoxy.

8. The compound according to claim 1, wherein X¹ is hydrogen or methyl.

9. The compound according to claim 1, wherein X² is methyl.

10. The compound according to claim 1, wherein Y is O, S, or S(O).

11. A plant growth regulating or seed germination promoting composition, comprising the compound according to claim 1, and an agriculturally acceptable formulation adjuvant.

12. A method for regulating the growth of plants at a locus, said method comprising applying to the locus a compound according to claim 1.

13. A method for promoting the germination of seeds, comprising applying to the seeds, or a locus containing the seeds, the compound according to claim 1.

14. A method for improving the nutrient uptake of a crop, comprising applying to the plant or locus thereof, the compound according to claim 1.

15. Use of a compound of Formula (I) according to claim 1 for promoting the germination of seeds and/or for regulating plant growth.

16. A seed comprising a compound of Formula (I) according to claim 1.

17. The compound according to claim 2, wherein R^{3a}, R^{3b}, R^{3c}, R^{3d} are all hydrogen.

18. A method for regulating the growth of plants at a locus, said method comprising applying to the locus a compound according to a plant growth regulating amount of a composition according to claim 12.

19. A method for promoting the germination of seeds, comprising applying to the seeds, or a locus containing the seeds, the compound according to a seed germination promoting amount of a composition according to claim 13.

20. A method for improving the nutrient uptake of a crop, comprising applying to the plant or locus thereof, the compound according to a plant growth regulating or seed germination promoting composition according to claim 14.

21. Use of a compound of Formula (I) according to the composition as defined in claim 11, for promoting the germination of seeds and/or for regulating plant growth

22. A seed comprising a compound of Formula (I) according to a composition according to claim 11.
